(19) United States
(12) Patent Application Publication
(10) Pub. No.: US 2023/0052124 A1
(43) Pub. Date: Feb. 16, 2023

(54) DELIVERY APPARATUS AND METHODS FOR IMPLANTING PROSTHETIC HEART VALVES

(71) Applicant: Edwards Lifesciences Corporation, Irvine, CA (US)

(72) Inventors: Oren Cohen, Kadima (IL); Elazar Levi Schwarcz, Netanya (IL); Ofir Witzman, Harish (IL); Denis Zhmakin, Haifa (IL)

(21) Appl. No.: 17/978,849

(22) Filed: Nov. 1, 2022

Related U.S. Application Data
(63) Continuation of application No. PCT/US2021/034134, filed on May 26, 2021.
(60) Provisional application No. 63/033,673, filed on Jun. 2, 2020.
(60) Provisional application No. 63/031,787, filed on May 29, 2020.

(51) Int. Cl. A61F 2/24; A61F 2/95

(52) U.S. Cl. CPC A61F 2/243; A61F 2/9517; A61F 2002/9505

(57) ABSTRACT

A delivery apparatus for controlling implantation of a prosthetic heart valve includes a handle housing and a release mechanism mounted on the handle housing. The release mechanism can be operably coupled to at least one actuation shaft. Actuation of the release mechanism can cause a distal end portion of the actuation shaft to be connected to or released from the prosthetic heart valve. The handle also includes an indicator tab configured to indicate whether the actuation shaft if connected to or released from the prosthetic heart valve.

### CROSS-REFERENCE TO RELATED APPLICATION

This application is a continuation of a PCT Application No. PCT/US2021/034134, filed May 26, 2021, which claims the benefit of U.S. Provisional Applications Nos. 63/031,787, filed May 29, 2020, and 63/033,673, filed Jun. 2, 2020, where each of above-referenced applications is incorporated herein by reference in its entirety.

### FIELD

The present disclosure relates to implantable, mechanically-expandable prosthetic devices, such as prosthetic heart valves, and to delivery apparatus and methods for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically-expandable” prosthetic heart valves. Mechanically-expandable prosthetic heart valves can provide one or more advantages over self-expandable and balloon-expandable prosthetic heart valves. For example, mechanically-expandable prosthetic heart valves can be expanded to various diameters. Mechanically-expandable prosthetic heart valves can also be compressed after an initial expansion (e.g., for repositioning and/or retrieval).

Despite these advantages, mechanically-expandable prosthetic heart valves can present several challenges. For example, it can be difficult to release a mechanically-expandable prosthetic heart valve from the delivery apparatus. Premature retraction of the delivery apparatus may result in displacement of the prosthetic heart valve. Accordingly, there is a need for improved delivery apparatus and methods for implanting mechanically-expandable prosthetic heart valves.

### SUMMARY

Described herein are prosthetic heart valves, delivery apparatus, and methods for implanting prosthetic heart valves. The disclosed delivery apparatus and methods can, for example, help to ensure that, after a prosthetic heart valve is deployed, the delivery apparatus is disengaged from the prosthetic heart valve before withdrawing the delivery apparatus. The delivery apparatus and methods disclosed herein are also relatively simple and/or easy to use. This can, for example, reduce the risk of causing displacement of the prosthetic heart valve during withdrawal of the delivery apparatus.

In one representative embodiment, a delivery apparatus for implanting a prosthetic heart valve is provided. The delivery apparatus can include a handle housing, a release mechanism mounted on the handle housing, and a gear assembly having one or more pinion gears and an indicator gear. Actuation of the release mechanism can cause rotation of the one or more pinion gears and the indicator gear. Each pinion gear can be connected to a proximal end portion of a corresponding actuation shaft. Rotation of the pinion gear can cause rotation of the corresponding actuation shaft such that a distal end portion of the actuation shaft can be connected to or released from the prosthetic heart valve. The indicator gear can be operably connected to an indicator tab. Rotation of the indicator gear can cause the indicator tab to move between a first position and a second position relative to the handle housing such that when the indicator tab is at the first position, the one or more actuation shafts are connected to the prosthetic heart valve, and when the indicator tab is at the second position, the one or more actuation shafts are released from the prosthetic heart valve.

In another representative embodiment, a delivery apparatus can include a handle and one or more actuation shafts. Each actuation shaft can have a proximal end portion connected to the handle and a distal end portion that is releasably couplable to the prosthetic heart valve. The handle can include a housing and a release mechanism mounted on the housing. Actuation of the release mechanism can cause the distal end portion of the actuation shafts to be connected to or released from the prosthetic heart valve. The handle can further include an indicator tab configured to indicate whether the one or more actuation shafts are connected to or released from the prosthetic heart valve.

Certain embodiments of the disclosure also concern an assembly including a prosthetic heart valve having one or more actuators and a delivery apparatus having a handle and one or more actuation shafts. Actuation of the actuators can cause radial expansion or compression of the prosthetic heart valve. Each actuation shaft can include a proximal end portion connected to the handle and a distal end portion that is releasably couplable to a corresponding actuator. The handle can include a release mechanism. Actuation of the release mechanism can cause the distal end portion of the actuation shafts to be connected to or released from the corresponding actuators. The handle can further include an indicator tab configured to indicate whether the one or more actuation shafts are connected to or released from the corresponding actuators.

Also disclosed herein is a method for implanting a prosthetic heart valve. The method can include deploying the prosthetic heart valve at a target location within a patient's body using a delivery apparatus. The delivery apparatus can include a handle and at least one actuation shaft that is releasably couplable to an actuator of the prosthetic heart valve. The method can further include radially expanding the prosthetic heart valve, releasing the actuation shaft from the actuator, and confirming release of the actuation shaft from the actuator based on an indicator on the handle.

Certain embodiments of the disclosure also concern a delivery apparatus for controlling implantation of a prosthetic heart valve. The delivery apparatus can include a handle housing and a release mechanism mounted on the handle housing. The release mechanism can be operably coupled to at least one actuation shaft, and actuation of the release mechanism can cause a distal end portion of the actuation shaft to be connected to or released from the prosthetic heart valve. The delivery apparatus can also include an indicator tab configured to indicate whether the actuation shaft if connected to or released from the prosthetic heart valve.

Also disclosed herein is a delivery apparatus for implanting a prosthetic heart valve. The delivery apparatus can include a handle having a release mechanism and at least one actuation shaft connected to the handle. The actuation shaft can be configured to be releasably connected to the prosthetic heart valve and cause radial expansion or compression of the prosthetic heart valve. Actuation of the release mechanism can cause a distal end portion of the actuation shaft to be connected to or released from the prosthetic heart valve. The release mechanism can be operatively connected to an indicator which is configured to indicate whether the actuation shaft is connected to or released from the prosthetic heart valve.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a delivery assembly comprising a mechanically-expandable prosthetic heart valve and a delivery apparatus.

FIG. 2 is a perspective view of the prosthetic heart valve.

FIG. 3 is another perspective view of the prosthetic heart valve without the valve structure and with the frame of the prosthetic heart valve in a radially expanded configuration.

FIG. 4 is a side view of the prosthetic heart valve in a radially compressed configuration.

FIG. 5 is a detail of an actuator of the prosthetic heart valve.

FIG. 6 is a cross-sectional view of the actuator of the prosthetic heart valve.

FIG. 7 is a side view of a proximal end portion of the delivery apparatus.

FIG. 8 is a side view of a distal end portion of the delivery apparatus.

FIG. 9 is a cross-sectional view of shafts of the delivery apparatus, taken along the line 9-9 as shown in FIG. 8.

FIG. 10 is a detail view of distal end portions of shafts of the delivery apparatus.

FIG. 11 is a detail view of the prosthetic heart valve released from the delivery apparatus.

FIG. 12 is a detail view of the prosthetic heart valve coupled to the delivery apparatus.

FIG. 13 is a side view of the prosthetic heart valve coupled to the delivery apparatus with the prosthetic heart valve in the radially expanded configuration.

FIG. 14 is a side view of the prosthetic heart valve coupled to the delivery apparatus with the prosthetic heart valve in the radially compressed configuration.

FIG. 15 is a side view of the distal end portion of the delivery assembly.

FIG. 16 depict a first step of an exemplary implantation procedure in which the prosthetic heart valve is implanted in a heart (shown in cross-section) with the delivery apparatus.

FIG. 17 depicts a second step of the exemplary implantation procedure in which the prosthetic heart valve is implanted in a heart (shown in cross-section) with the delivery apparatus.

FIG. 18 depicts a third step of the exemplary implantation procedure in which the prosthetic heart valve is implanted in a heart (shown in cross-section) with the delivery apparatus.

FIG. 19 depicts a fourth step of the exemplary implantation procedure in which the prosthetic heart valve is implanted in a heart (shown in cross-section) with the delivery apparatus.

FIG. 20A is a perspective view of a gear assembly of a release mechanism, according to one embodiment.

FIG. 20B is a close-up view of a portion of the gear assembly depicted in FIG. 20A.

FIG. 21 is a perspective view of a gear assembly of a release mechanism, according to another embodiment.

FIG. 22 is an end view of a gear assembly depicted in FIG. 21.

FIG. 23 is a cross-sectional view of an indicator assembly of the release mechanism depicted in FIG. 21.

### DETAILED DESCRIPTION

General Considerations

For purposes of this description, it should be understood that the disclosed embodiments can be adapted to deliver and implant prosthetic devices in any of the native annuluses of the heart (e.g., the pulmonary, mitral, and tricuspid annuluses) and/or cardiac vessels, and can be used with any of various delivery approaches (e.g., retrograde, antegrade, transseptal, transventricular, transatrial, etc.).

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved. The technologies from any example can be combined with the technologies described in any one or more of the other examples. In view of the many possible embodiments to which the principles of the disclosed technology may be applied, it should be recognized that the illustrated embodiments are only preferred examples and should not be taken as limiting the scope of the disclosed technology.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or”, as well as “and” and “or”.

### Exemplary Embodiments

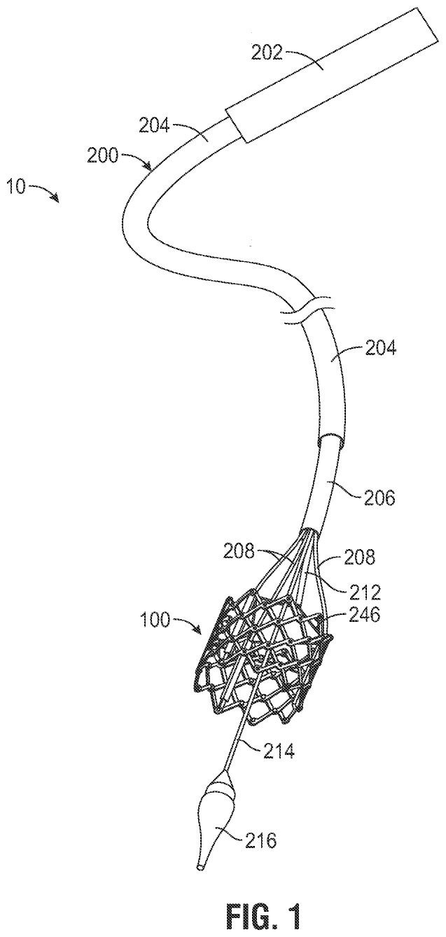

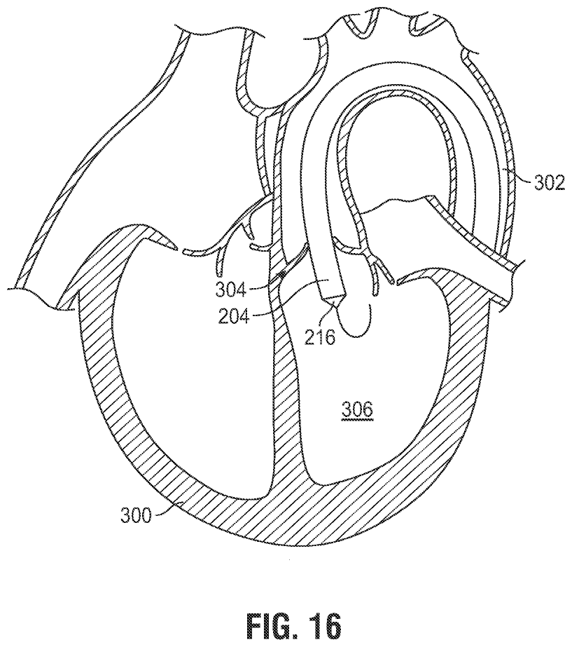

FIG. 1 shows a delivery assembly 10, according to one embodiment. In the illustrated embodiment, the delivery assembly 10 includes a prosthetic heart valve 100 and a delivery apparatus 200. The prosthetic valve 100 can be configured to replace a native heart valve (e.g., aortic, mitral, pulmonary, and/or tricuspid valves). As shown, the prosthetic valve 100 can be releasably coupled to a distal end portion of the delivery apparatus 200. The delivery apparatus 200 can be used to deliver and implant the prosthetic valve 100 in the native heart valve of a patient (see, e.g., FIGS. 16-19). Additional details regarding the prosthetic valve 100 and the delivery apparatus 200 are provided below.

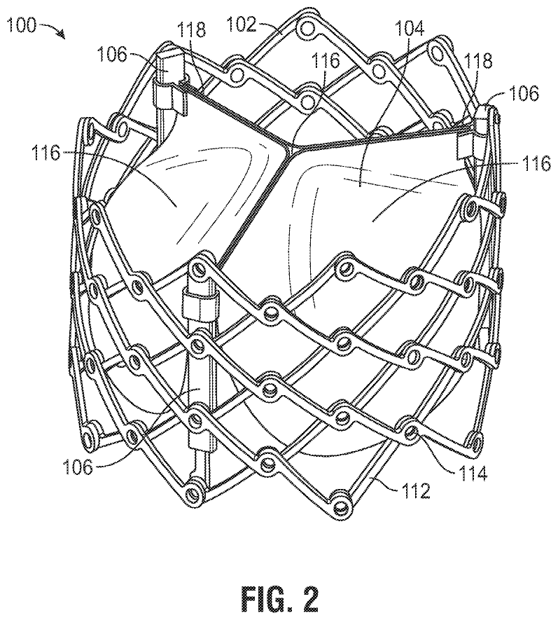

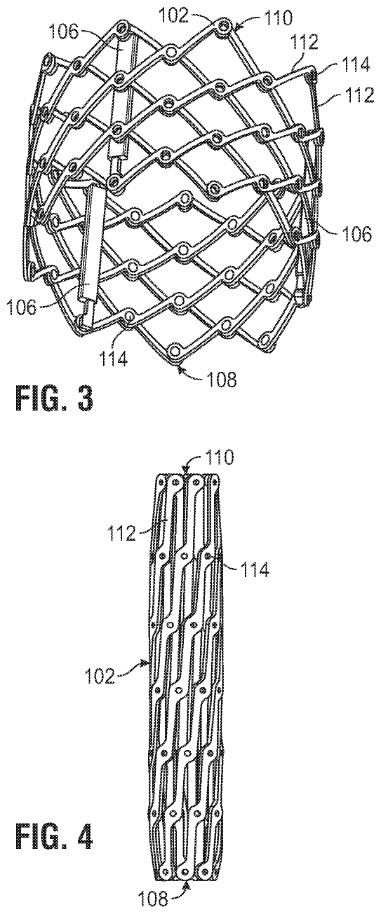

FIG. 2 shows the prosthetic valve 100. As shown, the prosthetic valve 100 can have three main components: a frame 102, a valve structure 104, and one or more actuators 106 (e.g., three actuators in the illustrated embodiment). The frame 102 (which can also be referred to as “a stent” or “a support structure”) can be configured for supporting the valve structure 104 and for securing the prosthetic valve 100 within a native heart valve. The valve structure 104 can be coupled to the frame 102 and/or to the actuators 106. The valve structure 104 can be configured to allow blood flow through the prosthetic valve 100 in one direction (i.e., antegrade) and to restrict blood flow through the prosthetic valve 100 in the opposition direction (i.e., retrograde). The actuators 106 can be coupled to the frame 102 and be configured to adjust expansion of the frame 102 to a plurality of configurations including one or more functional or expanded configurations (e.g., FIGS. 2-3), one or more delivery or compressed configurations (e.g., FIG. 4), and/or one or more intermediate configurations between the functional and delivery configurations. It should be noted that the valve structure 104 of the prosthetic valve 100 is not shown FIGS. 1 and 3-4 for purposes of illustration.

Referring to FIG. 3, the frame 102 of the prosthetic valve 100 has a first end 108 and a second end 110. In the illustrated embodiment, the first end 108 of the frame 102 is an inflow end and the second end 110 of the frame 102 is an outflow end. In other embodiments, the first end 108 of the frame 102 can be the outflow end and the second end 110 of the frame 102 can be the inflow end.

The frame 102 can be made of any of various suitable materials, including biocompatible metals and/or biocompatible polymers. Exemplary biocompatible metals from which the frame can be formed include stainless steel, cobalt chromium alloy, and/or nickel titanium alloy (which can also be referred to as “NiTi” or “nitinol”).

Referring still to FIG. 3, the frame 102 includes a plurality of interconnected struts 112 arranged in a lattice-type pattern. In FIG. 3, the frame 102 of the prosthetic valve 100 is in a radially expanded configuration, which results in the struts 112 of the frame 102 extending diagonally relative to a longitudinal axis of the prosthetic valve 100. In other configurations, the struts 112 of the frame 102 can be offset by a different amount than the amount depicted in FIG. 3. For example, FIG. 4 shows the frame 102 of the prosthetic valve 100 in a radially compressed configuration. In this configuration, the struts 112 of the frame 102 extend parallel (or at least substantially parallel) to the longitudinal axis of the prosthetic valve 100.

To facilitate movement between the expanded and compressed configurations, the struts 112 of the frame 102 can be pivotably coupled to one another at one or more pivot joints along the length of each strut. For example, each of the struts 112 can be formed with apertures at opposing ends and along the length of the strut. The frame 102 can include hinges at the locations where struts 112 overlap and are pivotably coupled together via fasteners such as rivets or pins 114 that extend through the apertures of the struts 112. The hinges can allow the struts 112 to pivot relative to one another as the frame 102 moves between the radially expanded and the radially compressed configurations, such as during assembly, preparation, and/or implantation of the prosthetic valve 100.

In some embodiments, the frame 102 can be constructed by forming individual components (e.g., the struts 112 and pins 114 of the frame 102) and then mechanically assembling and coupling the individual components together. In other embodiments, the struts are not coupled to each other with respective hinges but are otherwise pivotable or bendable relative to each other to permit radial expansion and contraction of the frame. For example, a frame can be formed (e.g., via laser cutting, electroforming or physical vapor deposition) from a single piece of material (e.g., a metal tube). Further details regarding the construction of frames and prosthetic valves are described in U.S. Publication Nos. 2018/0153689, 2018/0344456, and 2019/0060057, U.S. Application No. 62/869,948, and International Application No. PCT/US2019/056865, which are incorporated by reference herein. Additional examples of expandable prosthetic valves that can be used with the delivery apparatus disclosed herein are described in U.S. Publication Nos. 2015/0135506 and 2014/0296962, which are incorporated by reference herein.

Referring again to FIG. 2, the valve structure 104 of the prosthetic valve 100 can be coupled to the frame 102. The valve structure 104 can be configured to allow blood flow through the prosthetic valve 100 from the inflow end 108 to the outflow end 110 and to restrict blood from through the prosthetic valve 100 from the outflow end 110 to the inflow end 108. The valve structure 104 can include, for example, a leaflet assembly comprising one or more leaflets 116 (e.g., three leaflets in the illustrated embodiment).

The leaflets 116 of the prosthetic valve 100 can be made of a flexible material. For example, the leaflets 116 of the leaflet assembly can be made from in whole or part, biological material, bio-compatible synthetic materials, or other such materials. Suitable biological material can include, for example, bovine pericardium (or pericardium from other sources).

Referring to FIG. 2, the leaflets 116 can be arranged to form commissures 118 (e.g., pairs of adjacent leaflets), which can, for example, be mounted to respective actuators 106. Further details regarding prosthetic heart valves, including the manner in which the valve structure 104 can be coupled to the frame 102 of the prosthetic valve 100, can be found in U.S. Pat. Nos. 6,730,118, 7,393,360, 7,510,575, 7,993,394, and 8,652,202, and U.S. Publication No. 2018/0325665, which are incorporated by reference herein.

The valve structure 104 can be coupled to the actuators 106. For example, the commissures 118 of the valve structure 104 can be coupled to the housing members 122 of the actuators 106. Additional details regarding coupling the valve structure to the actuators can be found, for example, in U.S. Application No. 62/869,948.

As shown in FIG. 3, the actuators 106 of the prosthetic valve 100 can be mounted to and spaced circumferentially around the inner surface of the frame 102. The actuators 106 can be configured to, among other things, radially expand and/or radially compress the frame 102. The actuators 106 can also be configured to lock the frame 102 at a desired expanded configuration. Accordingly, the actuators 106 can also be referred to as “locking mechanisms.” Each of the actuators 106 can be configured to form a releasable connection with one or more respective actuation shafts of a delivery apparatus, as further described below.

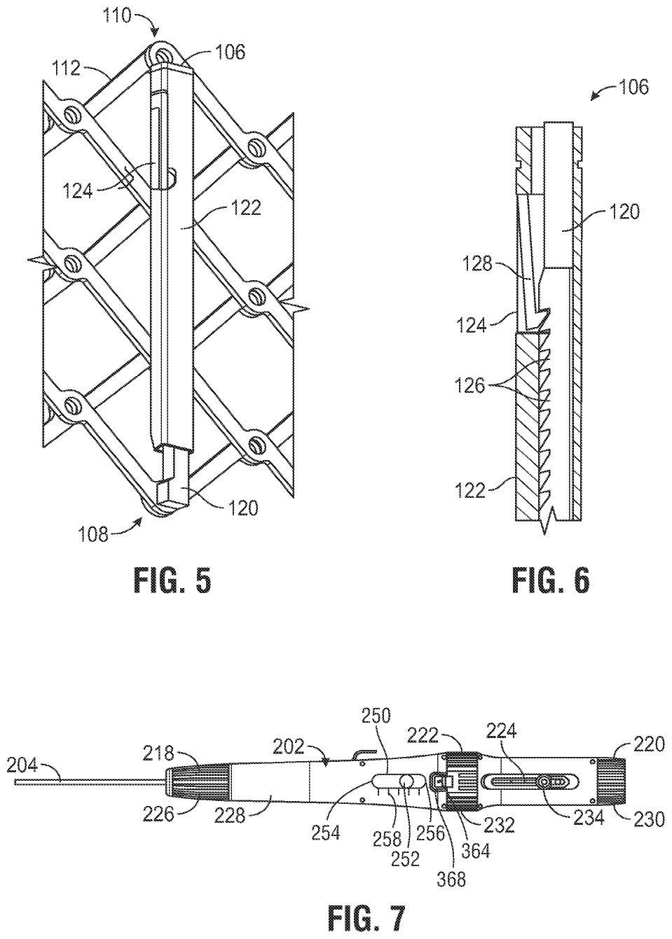

FIGS. 5-6 illustrate one exemplary embodiment of an actuator 106. As shown, the actuator 106 can include a rack member 120 (which can also be referred to as an “actuation member”), a housing member 122 (which can also be referred to as a “support member”), and a locking member 124. The rack members 120 can be coupled to the frame 102 of the prosthetic valve 100 at a first axial location (e.g., toward the inflow end 108 of the frame 102), and the housing members 122 can be coupled to the frame at a second axial location (e.g., toward the outflow end 110 of the frame 102). The rack members 120 can extend through and be axially movable relative to respective housing members 122. Thus, relative axial movement between the rack members 120 and the housing members 122 cab apply axially directed forces to the frame 102 and result in radial expansion/compression of the frame 102 as the struts 112 of the frame 102 pivot relative to each other about the pins 114. For example, moving the rack members 120 proximally (e.g., up in the orientation depicted in FIGS. 5-6) relative to the housing members 122 can radially expand the frame 102 (e.g., FIG. 3). Conversely, moving the rack members 120 distally (e.g., down in the orientation depicted in FIGS. 5-6) relative to the housing members 122 can radially compress the frame 102 (e.g., FIG. 4).

As shown in FIG. 6, one or more of the rack members 120 can include a segment with a plurality of teeth 126. The locking member 124 can be coupled to a respective housing member 122 and include a pawl 128 biased to engage the teeth 126 of the rack member 120. In this manner, the rack member 120 and the locking member 124 can form a ratchet-type mechanism that allows the rack member 120 to move proximally relative to the housing member 122 (thereby allowing expansion of the prosthetic valve 100) and that restricts the rack member 120 from moving distally relative to the housing member 122 (thereby restricting compression of the prosthetic valve 100).

In the illustrated embodiment, the locking member 124 is integrally formed with the housing member 122 as a unitary structure. In other embodiments, the locking member 124 and the housing member 122 can be formed as separate components that are coupled together (e.g., with fasteners, adhesive, welding, and/or other means for coupling).

It should be understood that the actuator 106 described above is merely one exemplary, but non-limiting, embodiment. For example, in other embodiments, the actuator can be configured to have a fixed inner member and a moveable outer member that annularly surrounds the inner member. To expand the prosthetic valve, the outer member can be configured to hold the inflow end (or outflow end) of the prosthetic valve stationary and the inner member can be configured to pull (or push) the outflow end (or inflow end) toward the inflow end (or outflow end) of the prosthetic valve. More generally, the actuator can be configured to have two members that can be moved axially relative to each other. In some embodiments, the two members can be arranged side-by-side instead of coaxially. To expand the prosthetic valve, one member can be configured to hold the inflow end (or outflow end) of the prosthetic valve stationary and the other member can be configured to pull (or push) the outflow end (or inflow end) toward the inflow end (or outflow end) of the prosthetic valve. Further, locking of the prosthetic valve can be implemented by means other than the ratchet-type mechanism, such as using an axially moveable locking nut as described in PCT Application PCT/US2020/013429, which is incorporated by reference herein.

In the illustrated embodiment, the prosthetic valve 100 includes three actuators 106. In other embodiments, a greater or fewer number of actuators can be used. For example, in one embodiment, the prosthetic valve can have one actuator. As another example, the prosthetic valve can have two actuators. In yet another example, a prosthetic valve can have 4-15 actuators.

Although not shown, the prosthetic valve 100 can also include one or more skirts or sealing members. For example, the prosthetic valve 100 can include an inner skirt mounted on the inner surface of the frame 102. The inner skirt can function as a sealing member to prevent or decrease perivalvular leakage, to anchor the leaflets 116 to the frame 102, and/or to protect the leaflets 116 against damage caused by contact with the frame 102 during crimping and during working cycles of the prosthetic valve 100. The prosthetic valve 100 can also include an outer skirt mounted on the outer surface of the frame 102. The outer skirt can function as a sealing member for the prosthetic valve by sealing against the tissue of the native valve annulus and thus reducing paravalvular leakage around the prosthetic valve. The inner and outer skirts can be formed from any of various suitable biocompatible materials, including any of various synthetic materials (e.g., PET) or natural tissue (e.g., pericardial tissue). The inner and outer skirts can be mounted to the frame using sutures, an adhesive, welding, and/or other means for attaching the skirts to the frame.

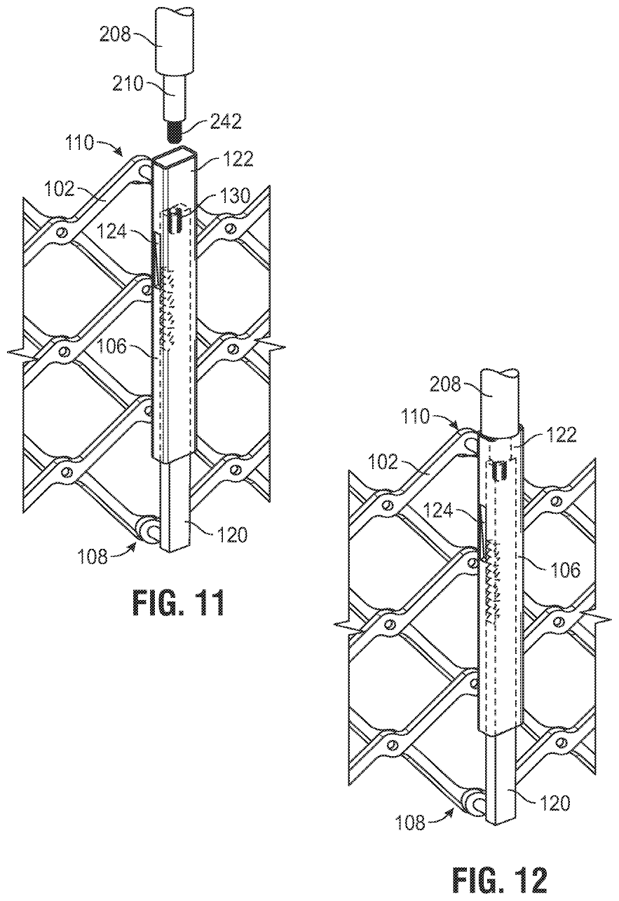

FIGS. 7-10 show the delivery apparatus 200 and its components, which can also be referred to as a “valve catheter” or a “delivery catheter.” As shown, the delivery apparatus 200 can include a handle 202, a first shaft 204, a second shaft 206, one or more support sleeves 208 (e.g., three in the illustrated embodiment), one or more actuation shafts 210 (e.g., three in the illustrated embodiment), an optional recompression shaft 212, a nosecone shaft 214, and a nosecone 216. The handle 202 can be configured for manipulating the shafts and sleeves relative to each other. The prosthetic heart valve 100 can be releasably coupled to the distal end portion of the delivery apparatus 200 (see, e.g., FIGS. 11-13), and the delivery apparatus 200 can be used for positioning the prosthetic valve 100, and/or for expanding, compressing, and locking the prosthetic valve 100 in a desired radially expanded configuration.

In the illustrated embodiment, the delivery apparatus 200 includes three pairs of support sleeves 208 and actuation shafts 210 (i.e., one pair of a support sleeve 208 and an actuation shaft 210 for each actuator 106 of the prosthetic valve 100). In other embodiments, the delivery apparatus 200 can have less than three (e.g., 1-2) or more than three (e.g., 4-15) pairs of support sleeves 208 and actuation shafts 210, depending on the number of actuators a prosthetic valve includes.

The handle 202 of the delivery apparatus 200 can have one or more mechanisms configured to move the shafts and sleeves relative to each other. For example, as shown in FIG. 7, the handle 202 includes a first mechanism 218, a second mechanism 220, a third mechanism 222, and/or a fourth mechanism 224.

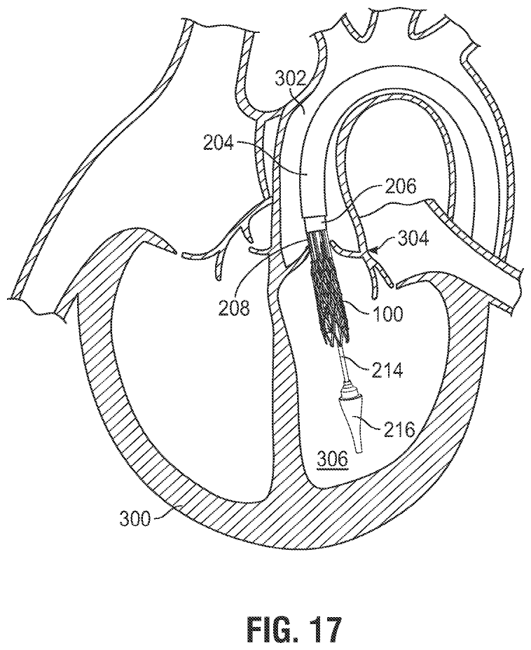

The first mechanism 218 of the handle 202 can be coupled to the first and second shafts 204, 206 and be configured to move the first and second shafts 204, 206 axially relative to each other. As further explained below, the first mechanism 218 of the handle 202 can be used to deploy the prosthetic valve 100 from the delivery capsule of the first shaft 204 (see FIG. 17). As such, the first mechanism 218 can be referred to as “a deployment mechanism.”

In the illustrated embodiment, the first mechanism 218 includes a first knob 226 configured for actuating the first mechanism 218. Although not shown, in other embodiments, the first mechanism 218 can include various other types of actuators configured for actuating the first mechanism 218, such as buttons, switches, etc. The first mechanism 218 can also include one or more other non-illustrated components (such as electric motors, rotatable shafts, drive screws, gear assemblies, etc.) configured to facilitate and/or restrict relative axial movement between the first and second shafts 204, 206. For example, the first mechanism 218 can be configured such that rotating the first knob 226 (and/or an electric motor) relative to a housing 228 of the handle 202 results in relative axial movement between the first and second shafts 204, 206.

The second mechanism 220 of the handle 202 can be coupled to the actuation shafts 210 and be configured to move the actuation shafts 210 axially relative to the support sleeves 208. When the prosthetic valve 100 is coupled to the delivery apparatus 200 via the actuation shafts 210, the second mechanism 220 of the handle 202 can be used to radially expand and/or compress the prosthetic valve 100, as further explained below. Accordingly, the second mechanism 220 can be referred to as “an actuation mechanism” and/or “an expansion mechanism.”

In the illustrated embodiment, the second mechanism 220 includes a second knob 230 configured for actuating the second mechanism 220. In other embodiments, the second mechanism 220 can include various other types of actuators. Although not shown, the second mechanism 220 can also include one or more additional components configured to facilitate and/or restrict relative axial movement of the actuation shafts 210 relative to the support sleeves 208. For example, the second mechanism 220 can include electric motors, drive screws, gear assemblies, and/or other components. In some embodiments, the second mechanism 220 can be configured such that rotating the second knob 230 (and/or an electric motor) relative to the housing 228 of the handle results in relative axial movement between the actuation shafts 210 and the support sleeves 208.

The third mechanism 222 of the handle 202 can also be coupled to the actuation shafts 210 and be configured to rotate the actuation shafts 210 relative to the support sleeves 208. In this manner, the third mechanism 222 can be used to simultaneously couple and release the actuation shafts 210 to/from the prosthetic valve 100, as further described below. Thus, the third mechanism 222 can be referred to as “a release mechanism” or “a coupling mechanism.”

In the illustrated embodiment, the third mechanism 222 includes a third knob 232 configured for actuating the third mechanism 222. In other embodiments, the third mechanism 222 can include various other types of actuators. The third mechanism 222 can also include one or more other components (e.g., a gear assembly and/or an electric motor) configured to facilitate and/or restrict relative rotational movement between the actuation shafts 210 and the support sleeves 208. For example, the third mechanism 222 can be configured such that rotating the third knob 232 relative to the housing 228 results in rotation of the actuation shafts 210 relative to the support sleeves 208.

The fourth mechanism 224 of the handle 202 can be coupled to the nosecone shaft 214 and be configured to move the nosecone shaft 214 and the nosecone 216 axially relative to the first and second shafts 204, 206. As such, the fourth mechanism 224 can be referred to as a “nosecone mechanism.”

In the illustrated embodiment, the fourth mechanism 224 includes a slider 234 configured for actuating the fourth mechanism 224. Although not shown, the fourth mechanism 224 can include various other components configured to facilitate and/or restrict relative axial movement of the nosecone shaft 214 and the first and second shafts 204, 206. For example, in some embodiments, the fourth mechanism 224 can include one or more biasing members (e.g., springs) configured to bias the nosecone shaft 214 to a pre-determined axial position relative to the first and second shafts 204, 206. In such embodiments, the slider 234 can be biased to a particular axial position relative to the housing 228 (e.g., to a proximal position). The nosecone shaft 214 can be moved axially relative to the first and second shafts 204, 206 by sliding the slider 234 relative to the housing 228 with sufficient force to overcome the opposing force of the biasing members. Upon release, the slider 234 can return to the biased position. In other embodiments, the fourth mechanism can include a rotatable knob, an electric motor, and/or a drive screw configured to convert relative rotational movement between the knob (and/or motor) and the housing into relative axial movement between the nosecone shaft and the first and second shafts.

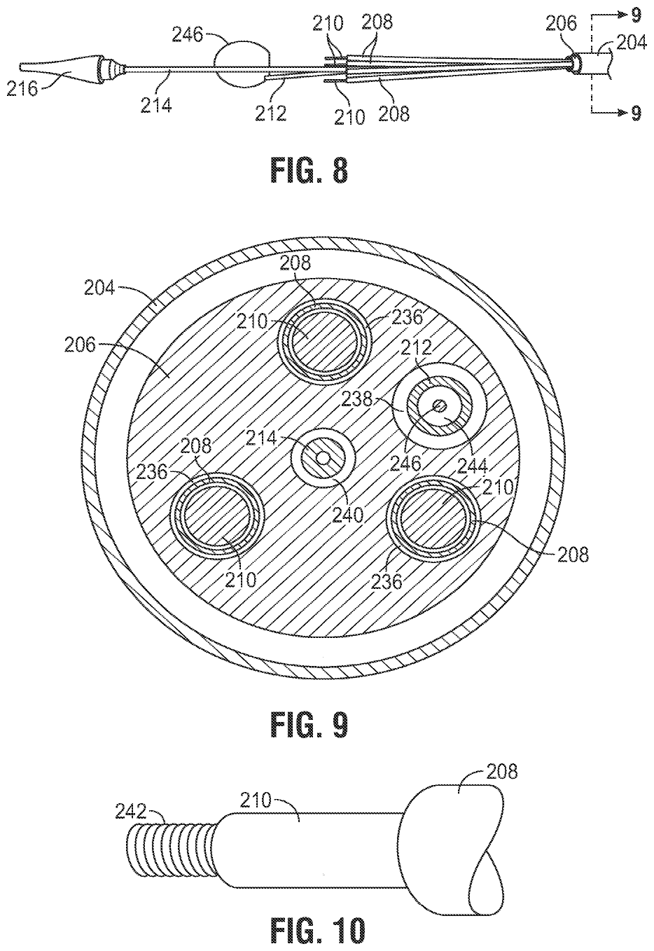

Referring now to FIGS. 7-8, a proximal end portion of the first shaft 204 can be coupled to and extends distally from the handle 202. The first shaft 204 can have a lumen for housing the second shaft 206 of the delivery apparatus 200. The distal end portion of the first shaft 204 can be configured to receive the prosthetic valve 100 in the radially compressed configuration (see FIGS. 14-17). As such, the first shaft 204 can be referred to as “a sheath” or “a delivery capsule”. Alternatively, the delivery capsule can be a separately formed component coupled to the distal end portion of the first shaft 204.

As shown in FIGS. 8-9, the second shaft 206 can extend coaxially through and be axially movable relative to the first shaft 204. The second shaft 206 can include a plurality of lumens extending axially therethrough and can thus be referred to as “a multi-lumen shaft.” For example, as shown in FIG. 9, the second shaft 206 can include one or more first lumens 236 (e.g., three in the illustrated embodiment) spaced circumferentially relative to each other. The first lumens 236 can be configured to receive respective actuation shafts 210 and/or support sleeves 208. In the illustrated embodiment, the first lumens 236 are evenly spaced relative to each other (i.e., spaced apart by about 120 degrees). In other embodiments, the first lumens 236 can be non-evenly spaced relative to each other.

In some embodiments, the second shaft 206 can also include one or more additional lumens. For example, as shown in FIG. 9, the second shaft 206 can include a recompression lumen 238 and a guidewire lumen 240. The guidewire lumen 240 can be radially centrally disposed in the second shaft 206. The recompression lumen 238 can be disposed radially outwardly relative to the guidewire lumen 240. In some embodiments, the recompression lumen 238 can be radially aligned with and/or spaced circumferentially relative to the first lumens 236.

In alternative embodiments, the second shaft 206 can have only one central lumen through which all the actuation shafts 210 and/or support sleeves 208 extend. Optionally, other shafts (e.g., the recompression shaft 212 and/or the nosecone shaft 214 described below) can also extend through such central lumen.

The support sleeves 208 can extend distally from respective first lumens 236 of the second shaft 206 and can be configured to contact the actuators 106 of the prosthetic valve 100 (see FIG. 12). The support sleeves 208 can be relatively more rigid than the actuation shafts 210. As such, the support sleeves 208 can be used to apply distally-directed forces to the housing members 122 of the actuators 106, which can oppose proximally-directed forces applied to the rack members 120 of the actuators 106 by the actuation shafts 210 of the delivery apparatus 200, thereby enabling expansion of the prosthetic valve 100 caused by relative axial movement between the rack members 120 and the housing members 122 of the actuators 106.

In the illustrated embodiment, the support sleeves 208 can be relative short tubes that are coupled to the distal end portion of the second shaft 206 but do not extend all the way through the second shaft 206 to the handle 202. The sleeves 208 can, in some instances, be secured to the inner surfaces of the second shaft 206 that define the first lumens 236 (e.g., via adhesive). In some embodiments, proximal end portions of the support sleeves 208 can be coupled to the handle 202, and the support sleeves 208 can extend through respective first lumens 236 of the second shaft 206 and beyond the distal end of the second shaft 206. In either instance, each of the support sleeves 208 can include a lumen configured to receive a respective actuation shaft 210, as shown in FIG. 9.

The actuation shafts 210 can extend distally from the handle 202, through respective first lumens 236 of the second shaft 206, and through the lumens of respective support sleeves 208. The distal end portions of the actuation shafts 210 can include mating features configured to releasably couple the actuation shafts to the actuators 106 of the prosthetic valve 100. For example, as shown in FIGS. 10-12, the distal end portions of the actuation shafts 210 can include external threads 242 configured to mate with corresponding internal threads 130 of the rack member 120 of the actuators 106. In other embodiments, the distal end portions of the actuation shafts 210 can have internal threads that are configured to mate with corresponding external threads of the rack member 120 of the actuators 106. Alternative coupling mechanism between the distal end portions of the actuation shafts 210 and the rack member 120 can be used.

In some embodiments, the actuation shafts 210 can be relatively flexible members. For example, the actuation shafts can be wires, cables, cords, sutures, etc. In other embodiments, the actuation shafts can be relatively rigid members, such as a rod. In other embodiments, the actuation shafts 210 can include one or more relatively flexible segments (e.g., at the distal end portions) and one or more relatively rigid segments (e.g., at the proximal end portions).

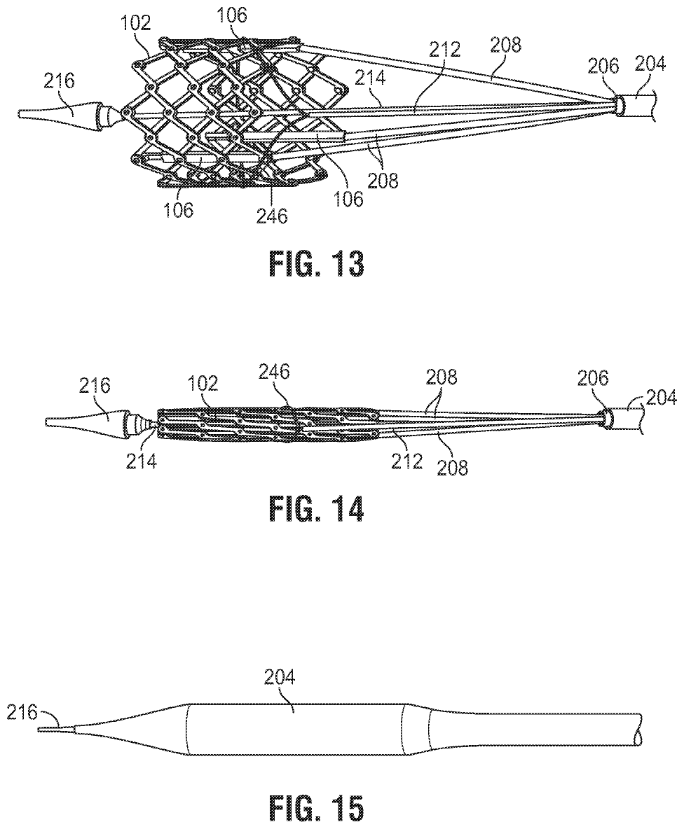

Referring to FIG. 8, the recompression shaft 212 can extend from the handle 202 through the recompression lumen 238 of the second shaft 206. As shown in FIG. 9, the recompression shaft 212 can have a lumen 244 through which a recompression member 246 (e.g., wire, cable, suture, etc.) extends. As shown in FIG. 13, the recompression member 246 can extend around the prosthetic valve 100 in a lasso-like manner. As such, the recompression member 246 can be used to recompress the prosthetic valve 100 by tensioning and thus constricting the recompression member 246 around the prosthetic valve 100.

The prosthetic valve 100 can be coupled to a distal end portion of the delivery apparatus 200 to form the delivery assembly (see FIGS. 11-13), and the delivery apparatus 200 can be used to implant the prosthetic valve 100 within a patient's body (see FIGS. 13-19). The prosthetic valve 100 can be coupled to the delivery apparatus 200 by positioning the delivery apparatus 200 in the configuration shown in FIG. 8. With the prosthetic valve 100 in the radially expanded configuration, the prosthetic valve 100 can be positioned over a proximal portion of the nosecone 216 and the nosecone shaft 214 and optionally within the loop of the recompression member 246, as shown in FIG. 13. The actuators 106 of the prosthetic valve 100 can be positioned adjacent the distal ends of the actuation shafts 210, as shown in FIG. 11. The actuation shafts 210 can then be inserted into the housing members 122 of the actuators 106 and threadably coupled to the rack members 120 of the actuators 106, as shown in FIG. 12.

With the prosthetic valve 100 releasably coupled to the delivery apparatus 200 (see FIG. 13), the prosthetic valve 100 can be radially compressed by actuating the actuators 106, for example, by tensioning the recompression member 246, and/or by inserting the prosthetic valve 100 and delivery apparatus 200 into a crimping device. Additional details about an exemplary crimping device for mechanically-expandable prosthetic valves can be found in U.S. Application No. 62/876,206, which is incorporated by reference herein. FIG. 14 shows the prosthetic valve 100 in a radially compressed configuration. The first shaft 204 of the delivery apparatus 200 can then be advanced over the second shaft 206 of the delivery apparatus 200 and the prosthetic valve 100 such that the prosthetic valve 100 is disposed within the lumen of the first shaft 204 and the distal end of the first shaft 204 abuts the nosecone 216, as shown in FIG. 15. This can be accomplished, for example, by actuating the first mechanism 218 of the handle 202.

The distal end portion of the delivery assembly 10 can then be inserted into a patient's vasculature, and the prosthetic valve 100 can be advanced to an implantation location using the delivery apparatus 200. For example, FIGS. 16-19 show an exemplary implantation procedure for implanting the prosthetic valve 100 within a patient's heart 300 using a transfemoral delivery procedure. In other embodiments, various other delivery procedures can be used, such as transventricular, transapical, transseptal, etc.

Referring to FIG. 16, the distal end portion of the delivery assembly 10 can be inserted into a patient's vasculature such that the first shaft 204 extends through the patient's aorta 302 and such that the nosecone 216 extends through the patient's native aortic annulus 304 and into the left ventricle 306 of the patient's heart 300. Turning to FIG. 17, the prosthetic valve 100 can be deployed from the first shaft 204 of the delivery apparatus 200 by actuating the first mechanism 218 of the handle 202, which moves the first shaft 204 of the delivery apparatus 200 proximally relative to the second shaft 206 of the delivery apparatus 200 (and/or moves the second shaft 206 distally relative to the first shaft 204). The first shaft 204 can be moved further proximally such that the support sleeves 208 are exposed from the distal end of the first shaft 204 (see, e.g., FIG. 14).

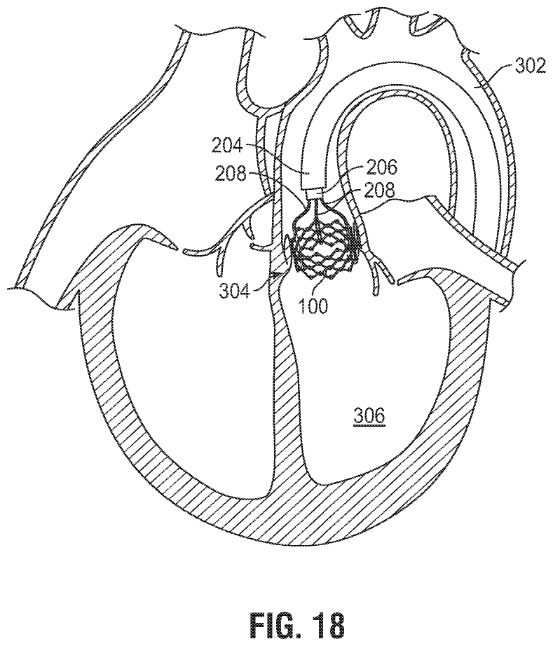

As shown in FIG. 18, the prosthetic valve 100 can then be radially expanded. This can be accomplished, for example, by actuating the second mechanism 220 of the handle 202 such that the actuation shafts 210 and the rack members 120 of the actuators 106 (which are coupled to the actuation shafts 210) can move proximally relative to the support sleeves 208 and the housing members 122 of the actuators 106 (which abut the distal ends of the support sleeves 208). When the prosthetic valve 100 is desirably positioned and secured within the native aortic annulus 304, the locking members 124 can engage the rack members 120 to retain the prosthetic valve 100 in the expanded state.

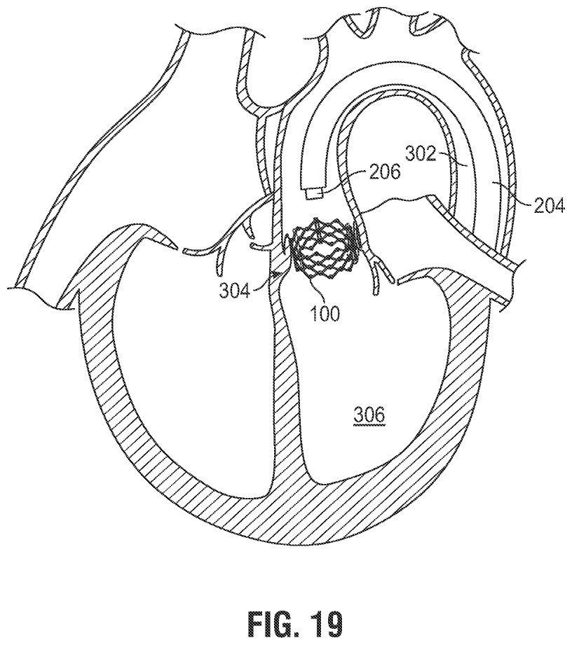

If re-positioning of the prosthetic valve is desired, the second mechanism 202 can be used to actuate the actuators 106 to radially compress the prosthetic valve 100. In lieu of or in addition to using the second mechanism 202, the prosthetic valve 100 can be recompressed and repositioned and/or retrieved using the recompression member 246. In some instances, the recompression member 246 can radially compress the prosthetic valve to a diameter that is smaller than is possible using only the actuators 106. It should be noted that, for purposes of illustration, the recompression shaft 212 and the recompression member 246 are not shown in FIGS. 17-18, and that the nosecone shaft 214 and the nosecone 216 are not shown in FIGS. 18-19.

Once expanded and secured, the prosthetic valve 100 can then be released from the delivery apparatus 200, as shown in FIG. 19. This can be accomplished by actuating the third mechanism 222 of the handle 202. This can rotate the actuation shafts 210 of the delivery apparatus 200 relative to the rack members 120 of the prosthetic valve 100, thereby de-coupling the threads 242 of the actuation shafts 210 from the threads 130 of the rack members 120. The actuation shafts 210, the support sleeves 208, and the second shaft 206 can then be withdrawn into the first shaft 204, and the delivery apparatus 200 can be removed from the patient's body.

Example embodiments of the handle 202, including certain exemplary embodiments of the first mechanism (i.e., the deployment mechanism) 218, the second mechanism (i.e., the expansion mechanism) 220, the third mechanism (i.e., the release mechanism) 222, and the fourth mechanism (i.e., the nosecone mechanism) 224, are described in Provisional U.S. Application No. 62/945,039, which is incorporated by reference herein.

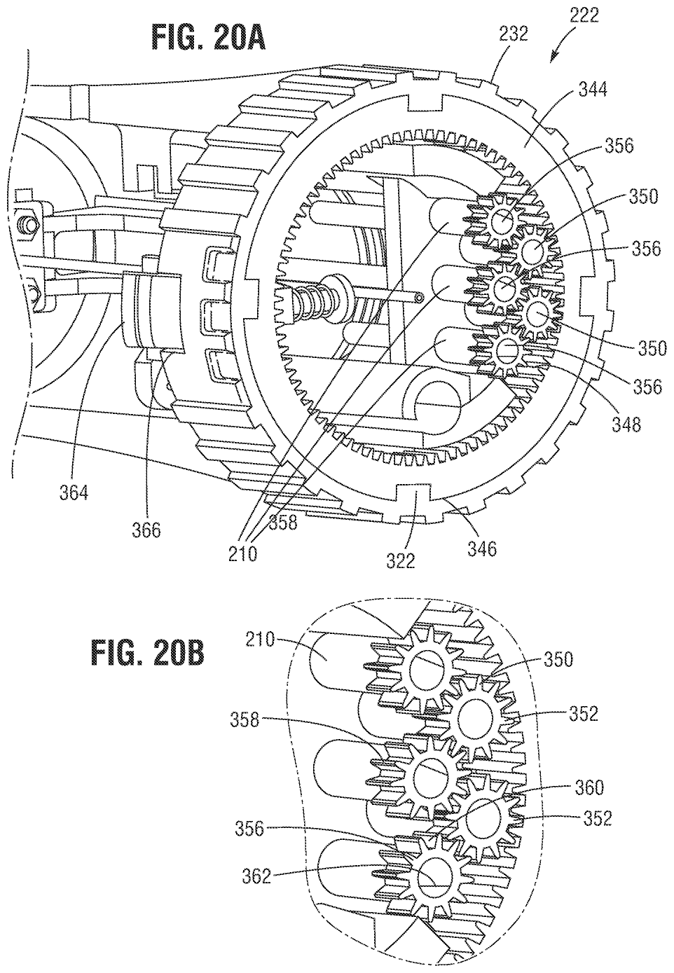

As an example, FIGS. 20A-B shows an enlarged view in perspective of some of the internal components of the handle 202 in the vicinity of the third mechanism 222. As shown, the third knob 232 can be configured to drive a gear train that include an annular driver gear 344 and a plurality of driven pinion gears 356 (three pinion gears are shown). The gear train can further include one or more idler gears 350 (two idler gears are shown). According to some embodiments, the third knob 232 can be attached to the annular driver gear 344, such that rotation of the third knob 232 in a clockwise or counterclockwise direction can cause rotation of the annular driver gear 344 in the same direction. According to some embodiments, the third knob 232 can have a plurality of knob mating projections 322, extending radially inward and received within respective driver gear mating recesses 346.

The annular driver gear 344 can have driver gear inner teeth 348 that mesh with idler gear teeth 352. The idler gear teeth 352 in turn can mesh with pinion gear teeth 358. As shown, the driver gear 344 can be meshed with, and configured to drive, two idler gears 350, one of which is meshed with, and configured to drive, two pinion gears 356, while the other idler gear 350 can be meshed with, and configured to drive, a single third pinion gear 356.

While two idler gears are shown, it will be understood that any number of idler gears may be used according to design requirements. While idler gears may be used to translate rotational movement to pinion gears that might be offset from the driver gear teeth 348, other embodiments may exclude idler gears, for example if the pinion gears 356 can be designed so as to directly contact and mesh with the driver gear teeth 348.

According to some embodiments, each pinion gear 356 can have a pinion gear bore 360, configured to receive an actuation shaft 210 therein. According to some embodiments, the gear bore 360 can have a non-circular feature, such as a flat edge 362, wherein the non-circular profile of the bore 360 matches a non-circular profile of a portion of the actuation shaft 210 extending therethrough. This non-circular profile allows the actuation shaft 210 to freely move axially in a proximal or a distal direction relative to the pinion gear 356, for example, when actuating the second mechanism 220 of the handle 202. Yet, as the pinion gear 356 is driven, for example by the driver gear 344 via an idler gear 350, the actuation shaft 210 can rotate therewith in the same direction.

When the third knob 232 is rotated in a specific direction (which can be clockwise or counterclockwise), all actuation shafts 210 can be rotated via the gear train (i.e., via the driver gear 344, the idler gears 350 and the driven pinion gears 356). Accordingly, the distal end portions of the actuation shafts 210 can be unthreaded and disengaged from the respective rack members 120.

According to some embodiments, the handle 202 can further include a safety knob 364 configured to slide into a recess 366 within the third rotatable knob 232, for example, along a slot 368 located on the housing 228 of the handle 202. In this position, the third rotatable knob 232 is prevented from rotating in any direction. This can serve as a safety measure, so as to prevent unintentional disengagement of the delivery apparatus from the prosthetic valve. Once the prosthetic valve is sufficiently expanded and positioned at the implantation site, the safety knob 364 may be pushed along the slot 368 away from the third knob safety recess 366, to enable manual rotation of the third knob 232.

According to some embodiments, rotation of the third rotatable knob 232 in a first direction can be translated to rotation of the distal end threads 242 of each actuation shaft 210 about its longitudinal axes, enabling it to disengage from the prosthetic valve, while rotation of the third rotatable knob 232 in an opposite, second direction, is prevented, which advantageously can avoid damage that might otherwise result from over-tightening the distal end threads 242 due to accidental rotation of the third rotatable knob 232 in the wrong direction.

According to some embodiments, the handle 202 can also include a ratcheting mechanism that allows the third rotatable knob 232 to rotate in one direction but prevents its rotation in the opposite direction. Further details of the ratcheting mechanism as well as other components of the release mechanism are described in Provisional U.S. Application No. 62/990,299, which is incorporated by reference herein.

The delivery apparatus 200 can be retracted from the patient's body only after the actuation shafts 210 are completely disengaged from the respective rack members 120. Otherwise, premature retraction of the delivery apparatus 200 (e.g., when the actuation shafts 210 are still engaged with the respective rack members 120) may result in displacement of the prosthetic heart valve 100 itself. In certain embodiments, the decoupling between the prosthetic heart valve 100 and the actuation shafts 210 can be verified under fluoroscopy or using other X-ray imaging modalities. For example, a clinician may visually inspect certain relatively opaque structures of the prosthetic heart valve 100 and the delivery apparatus 200 to verify that the actuation shafts 210 are completely decoupled from the rack members 120, e.g., by identifying a gap formed therebetween, or detecting spontaneous lateral movement of the distal ends of the actuation shafts 210. However, such method of verification is subjective and may be inaccurate. Accordingly, it is desirable to provide an objective indication and accurate feedback of whether the actuation shafts 210 are completely disengaged from the rack members 120 of the prosthetic heart valve 100.

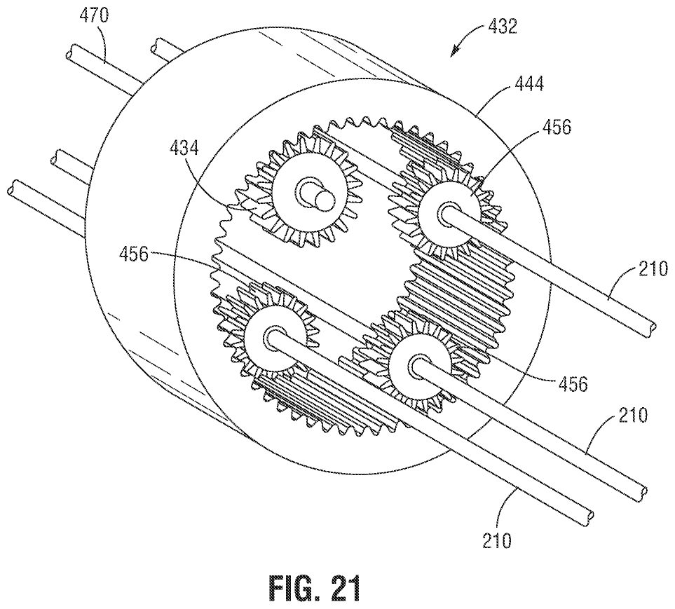

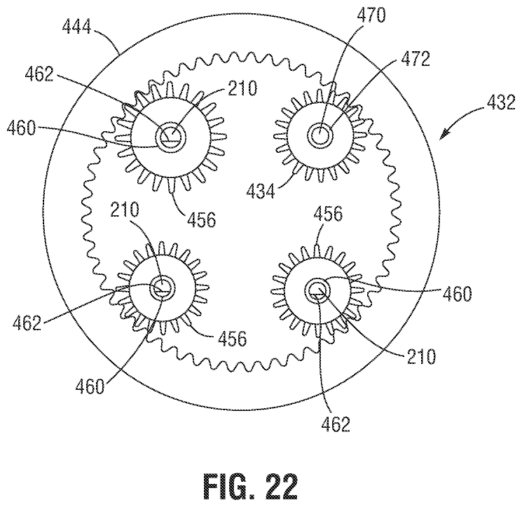

FIGS. 21-23 show another embodiment of release mechanism 222 including or being coupled to a gear assembly 432, which is configured to provide real-time feedback of engagement or disengagement between the actuation shafts 210 and the rack members 120 of the prosthetic heart valve 100. Specifically, as described below, the release mechanism 222 can include or be operatively coupled to an indicator configured to provide an objective and accurate feedback of the engagement/disengagement status between the actuation shafts 210 and the prosthetic heart valve 100. Although the release mechanism 222 and/or the gear assembly 432 are described below with respect to the delivery apparatus 200 and the prosthetic heart valve 100, it should be noted that the principles disclosed herein can be used to verify and/or monitor the engagement/disengagement status of any implantable medical devices and the respective delivery apparatuses.

As shown, the gear assembly 432 can include an annular driver gear 444 and one or more pinion gears 456 (three pinion gears are shown). In the depicted embodiment, the pinion gears 456 are configured to directly contact and mesh with the driver gear 444 such that rotation of the driver gear 444 can cause corresponding rotation of the one or more pinion gears 456. In other embodiments, the gear assembly 432 can also include one or more first idler gears (not shown), which are configured to mesh with the one or more pinion gears 456 and the driver gear 444 such that rotation of driver gear 444 can cause rotation of the one or more first idler gears, which in turn causes corresponding rotation of the one or more pinion gears 456.

The gear assembly 432 can also include an indicator gear 434. In the depicted embodiment, the indicator gear 434 is configured to directly contact and mesh with the driver gear 444 such that rotation of the driver gear 444 can cause corresponding rotation of the indicator gear 434. In other embodiments, the gear assembly 432 can also include at least one second idler gear (not shown), which is configured to mesh with the indicator gear 434 and the driver gear 444 such that rotation of driver gear 444 can cause rotation of the second idler gear, which in turn causes corresponding rotation of the indicator gear 434.

In some embodiments, the indicator gear 434 can have the same diameter as the one or more pinion gears 456. In some embodiments, the indicator gear 434 can have a different diameter than the one or more pinion gears 456. The number of teeth of the driver gear 444 to the number of teeth of a pinion gear 456 can define a first gear ratio. The number of teeth of the driver gear 444 to the number of teeth of the indicator gear 434 can define a second gear ratio. In some embodiments, the first gear ratio is the same as the second gear ratio such that one revolution of the driver gear 444 can cause the pinion gears 456 and the indicator gear 434 to have the same number of revolutions. In other embodiments, the first gear ratio is different than the second gear ratio such that one revolution of the driver gear 444 can cause the pinion gears 456 to have different number of revolutions (greater or less) than the indicator gear 434.

In some embodiments, each pinion gear 456 can be connected to a proximal end portion of a corresponding actuation shaft 210. Rotation of the pinion gear 456 can cause rotation of the corresponding actuation shaft such that a distal end portion (e.g., the thread 242) of the actuation shaft 210 can be connected to or released from the rack member 120 of the prosthetic heart valve 100. For example, each pinion gear 456 can have a pinion gear bore 460, configured to receive a corresponding actuation shaft 210 therein. In some embodiments, the gear bore 460 can have a non-circular feature, such as a flat edge 462, wherein the non-circular profile of the bore 460 matches a non-circular profile of a portion of the actuation shaft 210 extending therethrough. This non-circular profile allows the actuation shaft 210 to freely move axially in a proximal or a distal direction relative to the pinion gear 456, for example, when actuating the expansion mechanism 220 of the handle 202. Yet, as the pinion gear 456 is driven, for example by the driver gear 444, the actuation shaft 210 can rotate therewith in the same direction.

According to certain embodiments, the indicator gear 434 can be operably connected to an indicator which is configured to indicate whether the one or more actuation shafts 210 are connected to or released from the prosthetic heart valve 100. In the example embodiment described below, the indicator is an indicator tab 252 which is moveable between a first position and a second position relative to the housing 228 of the handle 202 such that when the indicator tab 252 is at the first position, the one or more actuation shafts 210 are connected to the prosthetic heart valve 100, and when the indicator tab 252 is at the second position, the one or more actuation shafts 210 are released from the prosthetic heart valve 100. In other embodiments, the indicator can be configured to provide other types of feedback to the user, such as in the form of audible sound, emitting light (e.g., via an LED), and/or tactile feedback, etc., to indicate whether the one or more actuation shafts 210 are connected to or released from the prosthetic heart valve 100.

As shown in FIGS. 21-23, the indicator gear 434 can be connected to a proximal end portion of an indicator shaft 470, and rotation of the indicator gear 434 can cause rotation of the indicator shaft 470 about its axial axis. For example, the indicator gear 434 can have an indicator gear bore 472 configured to receive a proximal end portion of the indicator shaft 470 therein. In some embodiments, the portion of the indicator shaft 470 inside the gear bore 472 can be matingly coupled to the indicator gear 434 such that when the indicator gear 434 is driven, for example by the driver gear 444, the indicator shaft 470 can rotate therewith in the same direction.

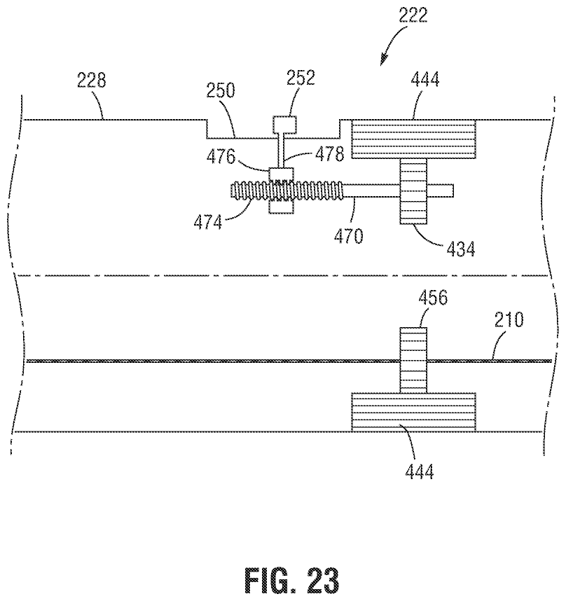

As shown in FIG. 23, the indicator shaft 470 can be operably connected to an indicator tab 252, which extends into a slot 250 on the housing 228 such that movement of the indicator tab 252 relative to the housing 228 can be visualized. For example, a distal end portion of the indicator shaft 470 can include external threads 474, which can be threadably coupled to internal threads of a nut 476. The nut 476 can be connected to the indicator tab 252 via an arm 478 extending between the nut 476 and the indicator tab 252. As best shown in FIG. 7, the tab 252 can be disposed in a slot 250 formed in the handle. The longitudinal sides of the slot prevent rotation of the tab 252 relative to the handle but allow sliding movement of the tab within the slot. Thus, rotation of the drive gear 444 drives rotation of the indicator gear 434, producing rotation of the indicator shaft 470. Since the tab 252 and the nut 476 are constrained against rotation by the dimensions of the slot 250, rotation of the indicator shaft 470 produces axial movement of the nut 476 along the indicator shaft 470, which causes corresponding axial movement of the indicator tab 252 relative to the housing 228.

In other embodiments, the indicator tab 252 can be configured to move in a non-axial direction. For example, indicator tab 252 can be operably coupled to the distal end portion of the indicator shaft 470 via a gear member (not shown) such that the rotation of the indicator shaft 470 can be converted into circumferential movement of the indicator tab 252. In yet further embodiments, the rotation of the indicator shaft 470 can be operatively coupled to other types of actuators that provide audial, visual, and/or tactile feedback to indicate the connection/release status between the actuation shafts 210 and the prosthetic heart valve 100.

According to certain embodiments, the release mechanism 222 can include an annular knob (e.g., knob 232) that is coupled to the driver gear 444 such that rotation of the knob in a clockwise or counterclockwise direction can cause rotation of the driver gear 444 in the same direction. In other embodiments, the release mechanism 222 can include an electric motor (not shown) coupled to the driver gear 444 such that actuation of the electric motor can cause rotation of the driver gear 444. In some embodiments, the electric motor can be actuated by pressing a button, flip a switch, etc.

Thus, by actuating the release mechanism 222 (e.g., rotating the knob 232), the driver gear 444 can be rotated in clockwise or counterclockwise direction, causing all pinion gears 456 and the indicator gear 434 to rotate simultaneously, together with the respective actuation shafts 210 and the indicator shaft 470 connected thereto. Rotating the actuation shafts 210 in a first angular direction (e.g., clockwise or counterclockwise) can cause the distal end portions of the actuation shafts 210 to be released from the respective rack members 120, while simultaneously rotating the indicator shaft 470 can cause the indicator tab 252 to move in a first axial direction (e.g., proximally or distally). Conversely, rotating the actuation shafts 210 in a second angular direction opposite to the first angular direction can cause the distal end portions of the actuation shafts 210 to be coupled to the respective rack members 120, while simultaneously rotating the indicator shaft 470 can cause the indicator tab 252 to move in a second axial direction opposite the first axial direction.

According to certain embodiments, the axial movement of the indicator tab 252 can be limited between a distal end 254 and a proximal end 256 of the slot 250 (see e.g., FIG. 7). In some embodiments, the housing 228 can have a plurality of graduation marks 258 or other indicia located adjacent to the slot 250 to indicate a precise location of the indictor tab 252 corresponding to a specific position of the actuation shafts between the connected and released states.

In some embodiments, the dimension of the slot 250 can be so configured that when the indicator tab 252 is moved to the distal end 254, the actuation shafts 210 are connected to the prosthetic heart valve 100, and when the indicator tab 252 is moved to the proximal end 256, the actuation shafts 210 are released from the prosthetic heart valve 100. Thus, as the release mechanism 222 is being actuated to release the shafts 210 from the prosthetic valve, the tab 252 moves in a proximal direction within the slot 250. In an alternative embodiment, the tab 252 can be configured to move from the proximal end 256 toward the distal end 254 of the slot as the release mechanism 222 is being actuated.

In some embodiments, one of the graduation marks 258 (e.g., a mark located in a mid-point between the distal end 254 and the proximal end 256) can be configured to mark a switch point. That is, when the indicator tab 252 is moved to the switch point, the distal end portions of the actuation shafts 210 are just about to be released from or connected to the respective rack members 120 of the prosthetic heart valve 100. Thus, from the switch point, further moving the indicator tab 252 toward the proximal end 256 (or distal end 254) can indicate the distal end portions of the actuation shafts 210 are moved further away (in a proximal direction) from the respective rack members 120, whereas further moving the indicator tab 252 toward the distal end 254 (or proximal end 256) can indicate the distal end portions of the actuation shafts 210 are threaded into (in a distal direction) the respective rack members 120.

As described above, after the prosthetic heart valve 100 has been deployed at a target location using the delivery apparatus 200, the prosthetic heart valve 100 can be radially expanded by actuating the expansion mechanism 220 on the handle 202. Once expanded and secured, the prosthetic heart valve 100 can be released from the delivery apparatus 200 and then the delivery apparatus 200 can be retracted from the patient's body.

Specifically, the prosthetic heart valve 100 can be released from the delivery apparatus 200 by actuating the release mechanism 222 on the handle 202. Actuation of the release mechanism 222 can activate the gear assembly 432, which in turn can cause rotation of the actuation shafts 210, thereby de-coupling the distal end portions of the actuation shafts 210 from the rack members 120 of the prosthetic heart valve 100. Actuation of the release mechanism 222 can also cause simultaneous rotation of the indicator shaft 470, thereby causing the indicator tab 252 to move axially along the slot 250. Release of the actuation shafts 210 from the rack members 120 can be confirmed by the position of the indicator tab 252 (e.g., moving past the switch point). To ensure the actuation shafts 210 are completely disengaged from the respective rack members 120, the release mechanism 222 can be further actuated until the indicator tab 252 is moved to a location (e.g., the proximal end 256 or distal end 254) which indicates that the distal end portions of the actuation shafts 210 are sufficiently separated from the respective rack members 120. Thus, the risk of displacing the prosthetic heart valve 110 during withdrawal of the delivery apparatus 200 can be reduced.

In view of the many possible embodiments to which the principles of the disclosure may be applied, it should be recognized that the illustrated embodiments are only examples and should not be taken as limiting the scope of the claims. Rather, the scope of the claimed subject matter is defined by the following claims and their equivalents.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for implantation of a prosthetic heart valve, the delivery apparatus comprising: a handle housing; a release mechanism mounted on the handle housing; and a gear assembly comprising one or more pinion gears and an indicator gear; wherein actuation of the release mechanism causes rotation of the one or more pinion gears and the indicator gear; wherein each pinion gear is connected to a proximal end portion of a corresponding actuation shaft, wherein rotation of the pinion gear causes rotation of the corresponding actuation shaft such that a distal end portion of the actuation shaft can be connected to or released from the prosthetic heart valve; wherein the indicator gear is operably connected to an indicator tab, wherein rotation of the indicator gear causes the indicator tab to move between a first position and a second position relative to the handle housing such that when the indicator tab is at the first position, the one or more actuation shafts are connected to the prosthetic heart valve, and when the indicator tab is at the second position, the one or more actuation shafts are released from the prosthetic heart valve.

Example 2. The delivery apparatus of any example herein, particularly example 1, wherein the indicator gear is connected to a proximal end portion of an indicator shaft, wherein a distal end portion of the indicator shaft is connected to the indicator tab, wherein rotation of the indicator gear causes rotation of the indicator shaft about its axial axis.

Example 3. The delivery apparatus of any example herein, particularly example 2, wherein the indicator tab is connected to a nut threadably coupled to the distal end portion of the indicator shaft, wherein rotation of the indicator shaft causes axial movement of the nut relative to the indicator shaft.

Example 4. The delivery apparatus of any example herein, particularly any one of examples 1-3, wherein the indicator tab extends into a slot on the handle housing such that movement of the indicator tab relative to the handle housing can be visualized.

Example 5. The delivery apparatus of any example herein, particularly example 4, wherein the handle housing comprises a plurality of graduation marks adjacent to the slot to indicate a location of the indictor tab between the first and second positions.

Example 6. The delivery apparatus of any example herein, particularly any one of examples 1-5, wherein the gear assembly comprises a driver gear operably coupled to the one or more pinion gears and the indicator gear such that rotation of the driver gear causes corresponding rotation of the one or more pinion gears and the indicator gear.

Example 7. The delivery apparatus of any example herein, particularly example 6, wherein the release mechanism comprises a knob coupled to the driver gear such that rotation of the knob relative to the handle housing causes corresponding rotation of the driver gear.

Example 8. The delivery apparatus of any example herein, particularly any one of examples 6-7, wherein the gear assembly comprises one or more first idler gears configured to mesh with the one or more pinion gears and the driver gear such that rotation of driver gear causes rotation of the one or more first idler gears, which in turn causes corresponding rotation of the one or more pinion gears.

Example 9. The delivery apparatus of any example herein, particularly any one of examples 6-8, wherein the gear assembly comprises a second idler gear configured to mesh with the indicator gear and the driver gear such that rotation of the driver gear causes rotation of the second idler gear, which in turn causes corresponding rotation of the indicator gear.

Example 10. The delivery apparatus of any example herein, particularly any one of examples 1-9, wherein the indicator gear has the same diameter as the one or more pinion gears.

Example 11. The delivery apparatus of any example herein, particularly any one of examples 1-9, wherein the indicator gear has a different diameter than the one or more pinion gears.

Example 12. A delivery apparatus for implanting a prosthetic heart valve, comprising:

a handle; and one or more actuation shafts, each having a proximal end portion connected to the handle and a distal end portion that is releasably couplable to the prosthetic heart valve; wherein the handle comprises a housing and a release mechanism mounted on the housing, wherein actuation of the release mechanism can cause the distal end portion of the actuation shafts to be connected to or released from the prosthetic heart valve; wherein the handle further comprises an indicator tab configured to indicate whether the one or more actuation shafts are connected to or released from the prosthetic heart valve.

Example 13. The delivery apparatus of any example herein, particularly example 12, wherein the indicator tab is moveable between a first position and a second position relative to the housing such that when the indicator tab is at the first position, the one or more actuation shafts are connected to the prosthetic heart valve, and when the indicator tab is at the second position, the one or more actuation shafts are released from the prosthetic heart valve.

Example 14. The delivery apparatus of any example herein, particularly example 13, wherein the indicator tab extends into a slot on the housing such that movement of the indicator tab relative to the housing can be visualized.

Example 15. The delivery apparatus of any example herein, particularly any one of examples 13-14, wherein the housing comprises a plurality of graduation marks between the first and second positions to indicate a connection status between the one or more actuation shafts and the prosthetic heart valve.

Example 16. The delivery apparatus of any example herein, particularly any one of examples 12-15, wherein the handle further comprises a gear assembly having one or more pinion gears and an indicator gear, wherein actuation of the release mechanism causes rotation of the one or more pinion gears and the indicator gear.

Example 17. The delivery apparatus of any example herein, particularly example 16, wherein the gear assembly comprises a driver gear operably coupled to the one or more pinion gears and the indicator gear such that rotation of the driver gear causes corresponding rotation of the one or more pinion gears and the indicator gear.

Example 18. The delivery apparatus of any example herein, particularly example 17, wherein the release mechanism comprises a knob coupled to the driver gear such that rotation of the knob relative to the housing causes corresponding rotation of the driver gear.

Example 19. The delivery apparatus of any example herein, particularly any one of examples 16-18, wherein the gear assembly comprises one or more first idler gears configured to mesh with the one or more pinion gears and the driver gear such that rotation of driver gear causes rotation of the one or more first idler gears, which in turn causes corresponding rotation of the one or more pinion gears.

Example 20. The delivery apparatus of any example herein, particularly any one of examples 16-19, wherein the gear assembly comprises a second idler gear configured to mesh with the indicator gear and the driver gear such that rotation of the driver gear causes rotation of the second idler gear, which in turn causes corresponding rotation of the indicator gear.

Example 21. The delivery apparatus of any example herein, particularly any one of examples 16-20, wherein each pinion gear is connected to the proximal end portion of a corresponding actuation shaft, wherein rotation of the pinion gear causes rotation of the corresponding actuation shaft such that the distal end portion of the actuation shaft can be connected to or released from the prosthetic heart valve.

Example 22. The delivery apparatus of any example herein, particularly any one of examples 16-21, wherein the indicator gear is operably connected to the indicator tab, wherein rotation of the indicator gear causes the indicator tab to move between the first position and the second position relative to the housing.

Example 23. The delivery apparatus of any example herein, particularly any one of examples 16-22, wherein the indicator gear is connected to a proximal end portion of an indicator shaft, wherein a distal end portion of the indicator shaft is connected to the indicator tab, wherein rotation of the indicator gear causes rotation of the indicator shaft about its axial axis.

Example 24. The delivery apparatus of any example herein, particularly example 23, wherein the indicator tab is connected to a nut threadably coupled to the distal end portion of the indicator shaft, wherein rotation of the indicator shaft causes axial movement of the nut relative to the indicator shaft.

Example 25. The delivery apparatus of any example herein, particularly any one of examples 12-24, further comprising one or more support sleeves, wherein each actuation shaft extends through a lumen of a corresponding support sleeve, wherein the distal end portion of each actuation shaft and a distal end portion of the corresponding support sleeve are configured to interface with a corresponding actuator of the prosthetic heart valve.

Example 26. The delivery apparatus of any example herein, particularly example 25, wherein the handle further comprises an expansion mechanism mounted on the housing, wherein the expansion mechanism is connected to the proximal end portion of each actuation shaft and a proximal end portion of each support sleeve, wherein actuation of the expansion mechanism can cause axial movement of the one or more actuation shafts relative to the corresponding support sleeves so as to actuate the actuators of the prosthetic heart valve for radial expansion or compression of the prosthetic heart valve.

Example 27. The delivery apparatus of any example herein, particularly any one of examples 25-26, further comprising an intermediate shaft, wherein the one or more actuation shafts and the corresponding support sleeves extend through one or more lumens of the intermediate shaft.

Example 28. The delivery apparatus of any example herein, particularly example 27, further comprising an inner shaft and a nose cone connected to a distal end portion of the inner shaft, wherein the inner shaft extends through a nosecone lumen of the intermediate shaft.

Example 29. The delivery apparatus of any example herein, particularly example 28, wherein the handle further comprises a nosecone mechanism mounted on the housing, wherein a proximal end portion of the inner shaft is connected to the nosecone mechanism such that actuation of the nosecone mechanism can cause axial movement of the inner shaft relative to the intermediate shaft.

Example 30. The delivery apparatus of any example herein, particularly any one of examples 27-29, further comprising an outer shaft, wherein the intermediate shaft extends through a lumen of the outer shaft, wherein a distal end portion of the outer shaft is configured to retain the prosthetic heart valve in a radially compressed configuration.

Example 31. The delivery apparatus of any example herein, particularly example 30, wherein the handle further comprises a deployment mechanism mounted on the housing, wherein the deployment mechanism is coupled to a proximal end portion of the intermediate shaft and a proximal end portion of the outer shaft, wherein actuation of the deployment mechanism can result in relative axial movement between the intermediate shaft and the outer shaft so as to allow the distal end portion of the outer shaft to cover or expose the prosthetic heart valve.

Example 32. An assembly, comprising: a prosthetic heart valve having one or more actuators, wherein actuation of the actuators can cause radial expansion or compression of the prosthetic heart valve; and a delivery apparatus comprising a handle and one or more actuation shafts; wherein each actuation shaft comprises a proximal end portion connected to the handle and a distal end portion that is releasably couplable to a corresponding actuator; wherein the handle comprises a release mechanism, wherein actuation of the release mechanism can cause the distal end portion of the actuation shafts to be connected to or released from the corresponding actuators; wherein the handle further comprises an indicator tab configured to indicate whether the one or more actuation shafts are connected to or released from the corresponding actuators.

Example 33. The assembly of any example herein, particularly example 32, wherein the indicator tab is moveable between a first position and a second position spaced apart from each other such that when the indicator tab is at the first position, the one or more actuation shafts are connected to the corresponding actuators, and when the indicator tab is at the second position, the one or more actuation shafts are released from the corresponding actuators.

Example 34. The assembly of any example herein, particularly example 33, wherein the indicator tab extends into a slot on the handle such that movement of the indicator tab can be visualized.

Example 35. The assembly of any example herein, particularly any one of examples 33-34, wherein the handle comprises a plurality of graduation marks between the first and second positions to indicate a degree of connection or separation between the one or more actuation shafts and the corresponding actuators.

Example 36. The assembly of any example herein, particularly any one of examples 32-35, wherein the handle further comprises a gear assembly having one or more pinion gears and an indicator gear, wherein actuation of the release mechanism causes rotation of the one or more pinion gears and the indicator gear.

Example 37. The assembly of any example herein, particularly example 36, wherein the gear assembly comprises a driver gear operably coupled to the one or more pinion gears and the indicator gear such that rotation of the driver gear causes corresponding rotation of the one or more pinion gears and the indicator gear.

Example 38. The assembly of any example herein, particularly example 37, wherein the release mechanism comprises a knob coupled to the driver gear such that rotation of the knob about its central axis causes corresponding rotation of the driver gear.

Example 39. The assembly of any example herein, particularly any one of examples 36-38, wherein the gear assembly comprises one or more first idler gears configured to mesh with the one or more pinion gears and the driver gear such that rotation of driver gear causes rotation of the one or more first idler gears, which in turn causes corresponding rotation of the one or more pinion gears.

Example 40. The assembly of any example herein, particularly any one of examples 36-39, wherein the gear assembly comprises a second idler gear configured to mesh with the indicator gear and the driver gear such that rotation of the driver gear causes rotation of the second idler gear, which in turn causes corresponding rotation of the indicator gear.

Example 41. The assembly of any example herein, particularly any one of examples 36-40, wherein each pinion gear is connected to the proximal end portion of a corresponding actuation shaft, wherein rotation of the pinion gear causes rotation of the corresponding actuation shaft such that the distal end portion of the actuation shaft can be connected to or released from the corresponding actuator.

Example 42. The assembly of any example herein, particularly any one of examples 36-41, wherein the indicator gear is operably connected to the indicator tab, wherein rotation of the indicator gear causes the indicator tab to move between the first position and the second position.

Example 43. The assembly of any example herein, particularly any one of examples 36-42, wherein the indicator gear is connected to a proximal end portion of an indicator shaft, wherein a distal end portion of the indicator shaft is connected to the indicator tab, wherein rotation of the indicator gear causes rotation of the indicator shaft about its axial axis.

Example 44. The assembly of any example herein, particularly example 43, wherein the indicator tab is connected to a nut threadably coupled to the distal end portion of the indicator shaft, wherein rotation of the indicator shaft causes axial movement of the nut relative to the indicator shaft.

Example 45. The assembly of any example herein, particularly any one of examples 32-44, wherein each actuator comprises an inner member received at least partially within an outer member, wherein relative axial movement between the inner member and the outer member causes radial expansion or compression of the prosthetic heart valve.

Example 46. The assembly of any example herein, particularly example 45, wherein the delivery apparatus further comprises one or more support sleeves, wherein each actuation shaft extends through a lumen of a corresponding support sleeve, wherein the distal end portion of each actuation shaft is configured to be releasably connected to the inner member, and a distal end portion of the corresponding support sleeve is configured to be releasably connected to the outer member.

Example 47. The assembly of any example herein, particularly example 46, wherein the distal end portion of each actuation shaft is configured to threadably coupled to a proximal end portion of the inner member and the distal end portion of the corresponding support sleeve is configured to abut a proximal end portion of the outer member such that axial movement of the actuation shaft relative to the corresponding support sleeve causes axial movement of the inner member relative to the outer member.

Example 48. The assembly of any example herein, particularly any one of examples 46-47, wherein the handle further comprises an expansion mechanism connected to the proximal end portion of each actuation shaft and a proximal end portion of each support sleeve, wherein actuation of the expansion mechanism can cause axial movement of the one or more actuation shafts relative to the corresponding support sleeves.

Example 49. The assembly of any example herein, particularly any one of examples 46-48, wherein the delivery apparatus further comprises an intermediate shaft, wherein the one or more actuation shafts and the corresponding support sleeves extend through one or more lumens of the intermediate shaft.

Example 50. The assembly of any example herein, particularly example 49, wherein the delivery apparatus further comprises an inner shaft and a nose cone connected to a distal end portion of the inner shaft, wherein the inner shaft extends through a nosecone lumen of the intermediate shaft.

Example 51. The assembly of any example herein, particularly example 50, wherein the handle further comprises a nosecone mechanism connected to a proximal end portion of the inner shaft such that actuation of the nosecone mechanism can cause axial movement of the inner shaft relative to the intermediate shaft.

Example 52. The assembly of any example herein, particularly any one of examples 49-51, wherein the delivery apparatus further comprises an outer shaft, wherein the intermediate shaft extends through a lumen of the outer shaft, wherein a distal end portion of the outer shaft is configured to retain the prosthetic heart valve in a radially compressed configuration.

Example 53. The assembly of any example herein, particularly example 52, wherein the handle further comprises a deployment mechanism coupled to a proximal end portion of the intermediate shaft and a proximal end portion of the outer shaft, wherein actuation of the deployment mechanism can result in relative axial movement between the intermediate shaft and the outer shaft so as to allow the distal end portion of the outer shaft to cover or expose the prosthetic heart valve.

Example 54. A method for implanting a prosthetic heart valve, the method comprising:

deploying the prosthetic heart valve at a target location within a patient's body using a delivery apparatus, the delivery apparatus comprising a handle and at least one actuation shaft that is releasably couplable to an actuator of the prosthetic heart valve; radially expanding the prosthetic heart valve; releasing the actuation shaft from the actuator; and confirming release of the actuation shaft from the actuator based on an indicator on the handle.

Example 55. The method of any example herein, particularly example 54, wherein deploying the prosthetic heart valve comprises navigating an outer shaft of the delivery apparatus through a patient's vasculature until a distal end portion of the outer shaft reaches the target location, wherein the distal end portion of the outer shaft is configured to retain the prosthetic heart valve in a radially compressed configuration.

Example 56. The method of any example herein, particularly example 55, wherein deploying the prosthetic heart valve comprises actuating a deployment mechanism on the handle, wherein actuating the deployment mechanism causes axial movement of the outer shaft relative to the actuation shaft so as to expose the prosthetic heart valve.

Example 57. The method of any example herein, particularly any one of examples 54-56, wherein radially expanding the prosthetic heart valve comprises axially moving an inner member of the actuator relative to an outer member of the actuator in a first direction.

Example 58. The method of any example herein, particularly example 57, further comprising radially compressing the prosthetic heart valve by axially moving the inner member relative to the outer member in a second direction that is opposite to the first direction.

Example 59. The method of any example herein, particularly any one of examples 57-58, wherein radially expanding the prosthetic heart valve comprises coupling a distal end portion of the actuation shaft to the inner member and abutting a distal end portion of a support sleeve against a proximal end portion of the outer member such that axial movement of the actuation shaft relative to the support sleeve causes corresponding axial movement of the inner member relative to the outer member, wherein the actuation shaft extends through a lumen of the support sleeve.

Example 60. The method of any example herein, particularly example 59, wherein radially expanding the prosthetic heart valve comprises actuating an expansion mechanism on the handle, wherein actuating the expansion mechanism causes axial movement of the actuation shaft relative to the support sleeve.

Example 61. The method of any example herein, particularly any one of examples 54-60, wherein releasing the actuation shaft comprises rotating the actuation shaft so as to threadably uncoupling the actuation shaft from the actuator.

Example 62. The method of any example herein, particularly example 61, wherein rotating the actuation shaft comprises rotating a pinion gear in the handle, wherein the pinion gear is coupled to a proximal end portion of the actuation shaft.

Example 63. The method of any example herein, particularly any one of examples 54-62, wherein the indicator comprises an indicator tab that is visible on the handle, wherein confirming release of the actuation shaft comprises moving the indicator tab from a first position to a second position, wherein when the indicator tab is at the first position, the actuation shaft is connected to the actuator, and when the indicator tab is at the second position, the actuation shaft is released from the actuator.

Example 64. The method of any example herein, particularly example 63, wherein moving the indicator comprises rotating an indicator shaft in the handle, wherein the indicator tab is connected to a nut threadably coupled to a distal end portion of the indicator shaft such that rotation of the indicator shaft causes axial movement of the nut relative to the indicator shaft.

Example 65. The method of any example herein, particularly example 64, wherein rotating the indicator shaft comprises rotating an indicator gear in the handle, wherein the indicator gear is coupled to a proximal end portion of the indicator shaft.

Example 66. The method of any example herein, particularly any one of examples 62 and 65, wherein rotating the indicator gear comprises rotating a driver gear in the handle, wherein the driver gear is operatively coupled to both the pinion gear and the indicator gear.

Example 67. The method of any example herein, particularly example 66, wherein rotating the driver gear comprises actuating a release mechanism on the handle, wherein actuating the release mechanism causes rotation of the driver gear.

Example 68. A delivery apparatus for controlling implantation of a prosthetic heart valve, the delivery apparatus comprising: a handle housing; a release mechanism mounted on the handle housing, wherein the release mechanism is operably coupled to at least one actuation shaft, wherein actuation of the release mechanism can cause a distal end portion of the actuation shaft to be connected to or released from the prosthetic heart valve; and an indicator tab configured to indicate whether the actuation shaft if connected to or released from the prosthetic heart valve.

Example 69. The delivery apparatus of any example herein, particularly example 68, wherein the indicator tab is moveable between a first position and a second position, wherein the first and second positions are so configured that when the indicator tab is at the first position, the actuation shaft is connected to the prosthetic heart valve, and when the indicator tab is at the second position, the actuation shaft is released from the prosthetic heart valve.

Example 70. The delivery apparatus of any example herein, particularly example 69, wherein the indicator tab extends into a slot on the handle such that movement of the indicator tab is visible by an operator of the handle.

Example 71. The delivery apparatus of any example herein, particularly any one of examples 68-70, further comprising a gear assembly having at least one pinion gear and an indicator gear, wherein actuation of the release mechanism causes rotation of the pinion gear and the indicator gear.

Example 72. The delivery apparatus of any example herein, particularly example 71, wherein the gear assembly comprises a driver gear operably coupled to the pinion gear and the indicator gear such that rotation of the driver gear causes corresponding rotation of the pinion gear and the indicator gear.

Example 73. The delivery apparatus of any example herein, particularly example 72, wherein the release mechanism comprises a knob coupled to the driver gear such that rotation of the knob about its central axis causes corresponding rotation of the driver gear.

Example 74. The delivery apparatus of any example herein, particularly any one of examples 71-73, wherein the gear assembly comprises a first idler gear configured to mesh with the pinion gear and the driver gear such that the first idler gear can transmit the rotation of driver gear to the pinion gear.

Example 75. The delivery apparatus of any example herein, particularly any one of examples 71-74, wherein the gear assembly comprises a second idler gear configured to mesh with the indicator gear and the driver gear such that the second idler gear can transmit the rotation of the driver gear to the indicator gear.

Example 76. The delivery apparatus of any example herein, particularly any one of examples 71-75, wherein the pinion gear is connected to a proximal end portion of the actuation shaft, wherein rotation of the pinion gear causes rotation of the actuation shaft such that the distal end portion of the actuation shaft can be connected to or released from the prosthetic heart valve.

Example 77. The delivery apparatus of any example herein, particularly any one of examples 71-76, wherein the indicator gear is operably connected to the indicator tab, wherein rotation of the indicator gear causes the indicator tab to move between the first position and the second position.

Example 78. The delivery apparatus of any example herein, particularly any one of examples 71-77, wherein the indicator gear is connected to a proximal end portion of an indicator shaft, wherein a distal end portion of the indicator shaft is connected to the indicator tab, wherein rotation of the indicator gear causes rotation of the indicator shaft about its axial axis.

Example 79. The delivery apparatus of any example herein, particularly example 78, wherein the indicator tab is connected to a nut threadably coupled to the distal end portion of the indicator shaft, wherein rotation of the indicator shaft causes axial movement of the nut relative to the indicator shaft.

Example 80. A delivery apparatus for implanting a prosthetic heart valve, comprising: a handle having a release mechanism; and at least one actuation shaft connected to the handle, wherein the actuation shaft is configured to be releasably connected to the prosthetic heart valve and cause radial expansion or compression of the prosthetic heart valve; wherein actuation of the release mechanism can cause a distal end portion of the actuation shaft to be connected to or released from the prosthetic heart valve; wherein the release mechanism is operatively connected to an indicator which is configured to indicate whether the actuation shaft is connected to or released from the prosthetic heart valve.

Example 81. The delivery apparatus of any example herein, particularly example 80, wherein the indicator comprises an indicator tab that is moveable between a first position and a second position, wherein the first and second positions are so configured that when the indicator tab is at the first position, the actuation shaft is connected to the prosthetic heart valve, and when the indicator tab is at the second position, the actuation shaft is released from the prosthetic heart valve.

Example 82. The delivery apparatus of any example herein, particularly any one of examples 80-81, wherein the release mechanism is coupled to a gear assembly comprising at least one pinion gear and an indicator gear, wherein actuation of the release mechanism causes rotation of the pinion gear and the indicator gear.

Example 83. The delivery apparatus of any example herein, particularly example 82, wherein the gear assembly comprises a driver gear operably coupled to the pinion gear and the indicator gear such that rotation of the driver gear causes corresponding rotation of the pinion gear and the indicator gear.

Example 84. The delivery apparatus of any example herein, particularly example 83, wherein the release mechanism comprises a knob coupled to the driver gear such that rotation of the knob about its central axis causes corresponding rotation of the driver gear.

Example 85. The delivery apparatus of any example herein, particularly any one of examples 82-84, wherein the gear assembly comprises a first idler gear configured to mesh with the pinion gear and the driver gear such that the first idler gear can transmit the rotation of driver gear to the pinion gear.

Example 86. The delivery apparatus of any example herein, particularly any one of examples 82-85, wherein the gear assembly comprises a second idler gear configured to mesh with the indicator gear and the driver gear such that the second idler gear can transmit the rotation of the driver gear to the indicator gear.

Example 87. The delivery apparatus of any example herein, particularly any one of examples 82-86, wherein the pinion gear is connected to a proximal end portion of the actuation shaft, wherein rotation of the pinion gear causes rotation of the actuation shaft such that the distal end portion of the actuation shaft can be connected to or released from the prosthetic heart valve.

Example 88. The delivery apparatus of any example herein, particularly any one of examples 82-87, wherein the indicator gear is operably connected to the indicator tab, wherein rotation of the indicator gear causes the indicator tab to move between the first position and the second position.

Example 89. The delivery apparatus of any example herein, particularly any one of examples 82-88, wherein the indicator gear is connected to a proximal end portion of an indicator shaft, wherein a distal end portion of the indicator shaft is connected to the indicator tab, wherein rotation of the indicator gear causes rotation of the indicator shaft about its axial axis.

Example 90. The delivery apparatus of any example herein, particularly example 89, wherein the indicator tab is connected to a nut threadably coupled to the distal end portion of the indicator shaft, wherein rotation of the indicator shaft causes axial movement of the nut relative to the indicator shaft.

Example 91. The delivery apparatus of any example herein, particularly any one of examples 80-90, further comprising a support sleeve, wherein the actuation shaft extends through a lumen of a support sleeve, wherein the distal end portion of the actuation shaft and a distal end portion of the corresponding support sleeve are configured to interface with a corresponding actuator of the prosthetic heart valve.

Example 92. The delivery apparatus of any example herein, particularly example 91, wherein the handle further comprises an expansion mechanism connected to a proximal end portion of the actuation shaft and a proximal end portion of support sleeve, wherein actuation of the expansion mechanism can cause axial movement of the actuation shaft relative to the support sleeve so as to actuate the actuator of the prosthetic heart valve for radial expansion or compression of the prosthetic heart valve.

Example 93. The delivery apparatus of any example herein, particularly any one of examples 92-93, further comprising an intermediate shaft, wherein the actuation shaft and the support sleeve extend through a first lumen of the intermediate shaft.

Example 94. The delivery apparatus of any example herein, particularly example 93, further comprising an inner shaft and a nose cone connected to a distal end portion of the inner shaft, wherein the inner shaft extends through a second lumen of the intermediate shaft.

Example 95. The delivery apparatus of any example herein, particularly example 94, wherein the handle further comprises a nosecone mechanism connected to a proximal end portion of the inner shaft such that actuation of the nosecone mechanism can cause axial movement of the inner shaft relative to the intermediate shaft.

Example 96. The delivery apparatus of any example herein, particularly any one of examples 93-95, further comprising an outer shaft, wherein the intermediate shaft extends through a lumen of the outer shaft, wherein a distal end portion of the outer shaft is configured to retain the prosthetic heart valve in a radially compressed configuration.

Example 97. The delivery apparatus of any example herein, particularly example 96, wherein the handle further comprises a deployment mechanism coupled to a proximal end portion of the intermediate shaft and a proximal end portion of the outer shaft, wherein actuation of the deployment mechanism can result in relative axial movement between the intermediate shaft and the outer shaft so as to allow the distal end portion of the outer shaft to cover or expose the prosthetic heart valve.

1. A delivery apparatus for implantation of a prosthetic heart valve, the delivery apparatus comprising:
a handle housing;
a release mechanism mounted on the handle housing; and
a gear assembly comprising one or more pinion gears and an indicator gear;
wherein actuation of the release mechanism causes rotation of the one or more pinion gears and the indicator gear;
wherein each pinion gear is connected to a proximal end portion of a corresponding actuation shaft, wherein rotation of the pinion gear causes rotation of the corresponding actuation shaft such that a distal end portion of the actuation shaft can be connected to or released from the prosthetic heart valve;
wherein the indicator gear is operably connected to an indicator tab, wherein rotation of the indicator gear causes the indicator tab to move between a first position and a second position relative to the handle housing such that when the indicator tab is at the first position, the one or more actuation shafts are connected to the prosthetic heart valve, and when the indicator tab is at the second position, the one or more actuation shafts are released from the prosthetic heart valve.

2. The delivery apparatus of claim 1, wherein the indicator gear is connected to a proximal end portion of an indicator shaft, wherein a distal end portion of the indicator shaft is connected to the indicator tab, wherein rotation of the indicator gear causes rotation of the indicator shaft about its axial axis.

3. The delivery apparatus of claim 2, wherein the indicator tab is connected to a nut threadably coupled to the distal end portion of the indicator shaft, wherein rotation of the indicator shaft causes axial movement of the nut relative to the indicator shaft.

4. The delivery apparatus of claim 1, wherein the indicator tab extends into a slot on the handle housing such that movement of the indicator tab relative to the handle housing can be visualized.

5. The delivery apparatus of claim 4, wherein the handle housing comprises a plurality of graduation marks adjacent to the slot to indicate a location of the indictor tab between the first and second positions.

6. The delivery apparatus of claim 1, wherein the gear assembly comprises a driver gear operably coupled to the one or more pinion gears and the indicator gear such that rotation of the driver gear causes corresponding rotation of the one or more pinion gears and the indicator gear.

7. The delivery apparatus of claim 6, wherein the release mechanism comprises a knob coupled to the driver gear such that rotation of the knob relative to the handle housing causes corresponding rotation of the driver gear.

8. A delivery apparatus for implanting a prosthetic heart valve, comprising:
a handle; and
one or more actuation shafts, each having a proximal end portion connected to the handle and a distal end portion that is releasably couplable to the prosthetic heart valve;
wherein the handle comprises a housing and a release mechanism mounted on the housing, wherein actuation of the release mechanism can cause the distal end portion of the actuation shafts to be connected to or released from the prosthetic heart valve;
wherein the handle further comprises an indicator tab configured to indicate whether the one or more actuation shafts are connected to or released from the prosthetic heart valve.

9. The delivery apparatus of claim 8, wherein the indicator tab is moveable between a first position and a second position relative to the housing such that when the indicator tab is at the first position, the one or more actuation shafts are connected to the prosthetic heart valve, and when the indicator tab is at the second position, the one or more actuation shafts are released from the prosthetic heart valve.

10. The delivery apparatus of claim 8, further comprising one or more support sleeves, wherein each actuation shaft extends through a lumen of a corresponding support sleeve, wherein the distal end portion of each actuation shaft and a distal end portion of the corresponding support sleeve are configured to interface with a corresponding actuator of the prosthetic heart valve.

11. The delivery apparatus of claim 10, wherein the handle further comprises an expansion mechanism mounted on the housing, wherein the expansion mechanism is connected to the proximal end portion of each actuation shaft and a proximal end portion of each support sleeve, wherein actuation of the expansion mechanism can cause axial movement of the one or more actuation shafts relative to the corresponding support sleeves so as to actuate the actuators of the prosthetic heart valve for radial expansion or compression of the prosthetic heart valve.

12. The delivery apparatus of claim 10, further comprising an intermediate shaft, wherein the one or more actuation shafts and the corresponding support sleeves extend through one or more lumens of the intermediate shaft.

13. The delivery apparatus of claim 12, further comprising an inner shaft and a nose cone connected to a distal end portion of the inner shaft, wherein the inner shaft extends through a nosecone lumen of the intermediate shaft.

14. The delivery apparatus of claim 13, wherein the handle further comprises a nosecone mechanism mounted on the housing, wherein a proximal end portion of the inner shaft is connected to the nosecone mechanism such that actuation of the nosecone mechanism can cause axial movement of the inner shaft relative to the intermediate shaft.

15. The delivery apparatus of claim 12, further comprising an outer shaft, wherein the intermediate shaft extends through a lumen of the outer shaft, wherein a distal end portion of the outer shaft is configured to retain the prosthetic heart valve in a radially compressed configuration.

16. The delivery apparatus of claim 15, wherein the handle further comprises a deployment mechanism mounted on the housing, wherein the deployment mechanism is coupled to a proximal end portion of the intermediate shaft and a proximal end portion of the outer shaft, wherein actuation of the deployment mechanism can result in relative axial movement between the intermediate shaft and the outer shaft so as to allow the distal end portion of the outer shaft to cover or expose the prosthetic heart valve.

17. A method for implanting a prosthetic heart valve, the method comprising:
deploying the prosthetic heart valve at a target location within a patient's body using a delivery apparatus, the delivery apparatus comprising a handle and at least one actuation shaft that is releasably couplable to an actuator of the prosthetic heart valve;
radially expanding the prosthetic heart valve;
releasing the actuation shaft from the actuator; and
confirming release of the actuation shaft from the actuator based on an indicator on the handle.

18. The method of claim 17, wherein releasing the actuation shaft comprises rotating the actuation shaft so as to threadably uncoupling the actuation shaft from the actuator.

19. The method of claim 17, wherein the indicator comprises an indicator tab that is visible on the handle, wherein confirming release of the actuation shaft comprises moving the indicator tab from a first position to a second position, wherein when the indicator tab is at the first position, the actuation shaft is connected to the actuator, and when the indicator tab is at the second position, the actuation shaft is released from the actuator.

20. The method of claim 19, wherein moving the indicator comprises rotating an indicator shaft in the handle, wherein the indicator tab is connected to a nut threadably coupled to a distal end portion of the indicator shaft such that rotation of the indicator shaft causes axial movement of the nut relative to the indicator shaft.
